(19) United States
(12) Patent Application Publication
(10) Pub. No.: US 2024/0261419 A1
(43) Pub. Date: Aug. 8, 2024

(54) PSILOCYBIN AND PSILOCIN CONJUGATES FOR TREATMENT OF MENTAL ILLNESSES

(71) Applicant: LONDON PHARMACEUTICALS AND RESEARCH CORPORATION, London (CA)

(72) Inventors: Collin Clarke, London (CA); Mahmoud Mohamed Abdrabo Moustafa, London (CA); Abdelrahman S. Mayhoub, Giza (EG)

(21) Appl. No.: 18/560,206

(22) PCT Filed: May 10, 2022
(86) PCT No.: PCT/CA2022/050733

§ 371 Date: Nov. 10, 2023

Related U.S. Application Data
(60) Provisional application No. 63/186,497, filed on May 10, 2021.

(51) Int. Cl. A61K 47/55; A61K 47/54

(52) U.S. Cl. CPC A61K 47/55; A61K 47/54; A61K 47/542; A61K 47/545; A61K 47/548

(57) ABSTRACT

New psilocybin and psilocin conjugates and their soluble salts with synergistic or additive therapeutic agents and stable formulations thereof, as well as their medical applications. The synergistic or additive conjugates may be used as drugs or prodrugs for treating various mental illness conditions related to the modulation or biased modulation of serotonin and related receptors, including but not limited to neurodegenerative disorders, depression, anxiety, obsessive compulsive disorder (OCD), cluster headaches, neuropathic pain and addiction.

### FIELD OF THE INVENTION

The present invention relates to the field of medicinal chemistry and, in particular, to conjugates of psilocin and its pro-drug psilocybin, their salts and therapeutic uses for treating or preventing psychological or neurological disorders.

### BACKGROUND

Existing therapies for mental illness seldom lead to clinical remission and frequently yield significant side effects. Furthermore, they have a delay in therapeutic onset and typically require a trial of multiple drugs before one is deemed effective. The economic burden of mental illness in Canada is estimated at $51 billion per year. This is exemplified by the Lancet Commission report citing an estimate that psychiatric disorders will cost the world US $16 Trillion by 2030. Therefore, identifying novel therapeutic strategies is of paramount importance to public health. Ideally, the next generation of psychiatric medications will have a low number needed to treat, fast onset of therapeutic action and minimal acceptable side effects.

Psilocybin, and its active form psilocin, can be classified as substituted dialkyl tryptamine derivatives and are the main chemical substances isolated from the genus Psilocybe (psychedelic mushrooms, which are an informal group including various species of mushrooms that contain psilocybin, psilocin or other related substances). They are emerging as an alternative therapy for mental illnesses such as post-traumatic stress disorder (PTSD), addiction, obsessive compulsive disorder (OCD), anxiety, Parkinson's disease, dementia, and depression. Aside from being a Schedule I substance and illegal to use since 1970, there are many fundamental challenges that prevented psilocybin from reaching the market as a medicinal product including: a) highly variable and subjective pharmacological actions: b) behavioral toxicity (e.g. psychosis); c) tachyphylaxis (loss of benefits after repeated doses): d) unknown dose-response relationship and duration of action.

Chemical structures of psilocybin and psilocin.

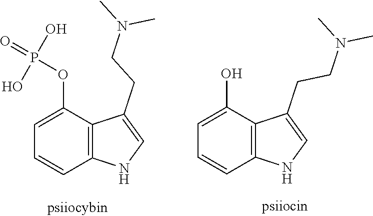 [SMILES: CN(C)CCc1c[nH]c2cccc(OP(=O)(O)O)c12 ; CN(C)CCc1c[nH]c2cccc(O)c12]

On the other hand, there are known clinical benefits of psilocybin and/or psilocin in combinations with cannabinoids like cannabidiol (CBD) or tetrahydrocannbidivarin (THC-V) either by separate, sequential, or simultaneous administration. For example, WO2018/135943A1 of Procare Beheer B.V. and US20180221396A1 of Caamtech LLC, disclose a combination therapy and compositions comprising individual therapeutic agents including psilocybin, a cannabinoid and/or terpenes for regulating a neurotransmitter receptor (e.g., a serotonin receptor) and prevention or treatment of psychological or brain disorders. Although promising, this therapeutic approach is hampered by the fact that these drugs exhibit substantially different pharmacokinetic profiles, so they do not reach their target tissues at the same time. In particular, psilocy bin reached its maximum plasma level after 90 mins, while it takes 4-6 hours for CBD to reach its maximum plasma level. The same remarkable difference was also observed in other PK parameters such as volume of distribution, rate of metabolism and elimination. Having two synergistically acting or additive molecules with different PK is a known major challenge in drug discovery and development.

To minimize the limitations in the prior art, there exists a demand for new synergistic and effective therapies with optimized physicochemical, pharmacokinetic (PK) and pharmacodynamic (PD) properties to manage mental illnesses.

### SUMMARY OF THE INVENTION

The psilocybin and psilocin compounds, according to the present invention, are labile conjugates of psilocybin and psilocin derivatives and salts with other synergistic or additive therapeutic agents. These new conjugates aim to deliver multiple therapeutic benefits via more than one mechanism of action. This is achieved by combining a psilocybin derivative with a different therapeutic agent with synergistic or additive effects using a hydrolysable spacer. The conjugates are sensitive to enzymatic or chemical hydrolysis within the animal or human body, to release the parent psilocybin derivative and the synergistic or additive therapeutic agent(s) thereby modulating their respective target receptors and tissues.

In some illustrative embodiments, this invention pertains to a compound having one of formulas 1-6 or pharmaceutically acceptable salts, hydrates, or solvates thereof, wherein:

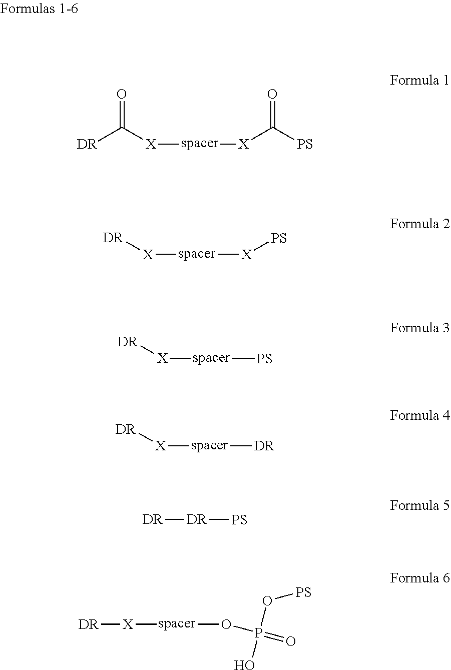

and wherein: DR is an additive or synergistic drug; PS is psilocybin, a psilocin derivative, or another related phosphorylated mushroom alkaloid; and each X is, independently, a linear, cyclic or branched alkyl carbon chain, with or without heteroatom(s), phosphate, phosphonate, substituted phosphate or phosphonate, oxygen, sulfur with any oxidation status, NH, or substituted nitrogen.

In another embodiment, PS is any psilocybin psilocin derivative or metabolite or another related phosphorylated mushroom alkaloid, including natural, synthetic or semisynthetic derivatives, preferably psilocybin and psilocin, or other analogues baeocystin, norbaeocystin, bufotenine, and aeruginascin.

In another embodiment, the natural phosphorylated mushroom alkaloids are:

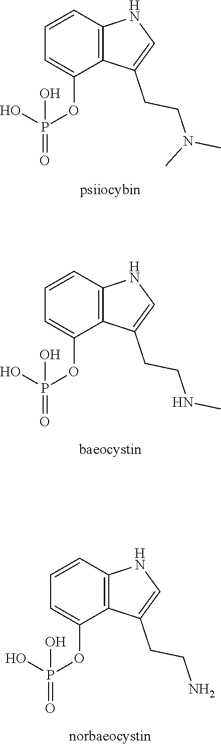 [SMILES: CN(C)CCc1c[nH]c2cccc(OP(=O)(O)O)c12 ; CNCCc1c[nH]c2cccc(OP(=O)(O)O)c12 ; NCCc1c[nH]c2cccc(OP(=O)(O)O)c12]

In another embodiment, the synthetic or semisynthetic phosphorylated related mushrooms alkaloids:

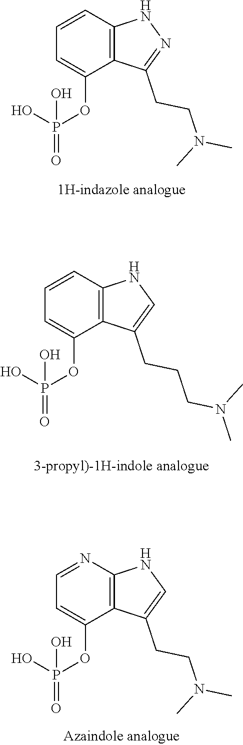 [SMILES: CN(C)CCc1n[nH]c2cccc(OP(=O)(O)O)c12 ; CN(C)CCCc1c[nH]c2cccc(OP(=O)(O)O)c12 ; CN(C)CCc1c[nH]c2nccc(OP(=O)(O)O)c12]

In another embodiment, DR is an additive or synergistic drug or its metabolite selected from pregabalin, gabapentin, topiramate, morphine, levodopa, selective serotonin reuptake inhibitor (SSRI) (e.g., citalopram), amisulpride, lurasidone, paliperidone, paliperidone palmitate, tapentadol, pentazocine, carbidopa, nalbuphine, risperidone, ziprasidone, perospirone, doxorubicin, melperone, aripiprazole, brexpiprazole, cariprazine, olanzapine, quetiapine, fluoxetine, calcitonin, pseudoephedrine, piracetam, levetiracetam, sitagliptin, silodosin, hydrochlorothiazide, ezetimibe, propranolol, atenolol, nadolol, pindolol, sotalol, timolol, penbutolol, oxprenolol, carvedilol, carteolol, bucindolol, acebutanol, betaxolol, esmolol, nebivolol, bisoprolol, celiprolol, metoprolol, azelnidipine, barnidipine, manidipine, lercandipine, efonidipine, benidipine, brimonidine, bortezomib, ledipasvir, daclatasvir, ombitasvir, elbasvir, lamivudine, dopamine, 5-hydroxytryptamine, levodopa, baclofen, pramipexole, ropinirole, rotigotine, apomorphine, tacrine, rivastigmine, donepezil, galantamine, vigabatrin, lamotrigine, tiagabine, amitriptyline, nortriptyline and histamine or a cannabinoid. Preferably, it is a cannabinoid.

In another embodiment, DR is one or more cannabinoids selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. Preferably, DR is CBD or THC-V.

In another embodiment, DR is one or more cannabinoid derivatives or metabolites, including natural, synthetic, or semisynthetic derivatives.

In another embodiment, X is, independently, a linear, cyclic or branched alkyl carbon chain, with or without heteroatom(s), phosphate, phosphonate, substituted phosphate or phosphonate, oxygen, sulfur with any oxidation status, NH, or substituted nitrogen. The X moieties may be identical or not.

In another embedment the spacer is a linear, branched, or cyclic alkane, alkene or alkyne, amino acid, acetal, ketal, peptide, aryl, heteroaryl, or heterocycloalkyl group, each of which is optionally substituted.

It is appreciated herein that the compounds described herein may be used alone or in combination with other compounds that may be therapeutically effective by the same or different modes of action. In addition, it is appreciated herein that the compounds described herein may be used in combination with other compounds that are administered to treat other symptoms of psychiatric disorders, such as compounds administered to relieve pain, nausea, vomiting, and the like.

### DESCRIPTION OF THE INVENTION

The psilocybin or psilocin conjugates or any of their derivatives or metabolites, according to the present invention, are two molecules connected directly through spacer(s) by covalent bond(s).

In some illustrative embodiments, this invention pertains to compounds having formula 1, or pharmaceutically acceptable salts, hydrates, or solvates thereof, wherein:

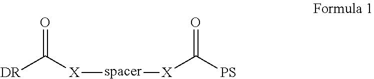

and wherein: DR is an additive or synergistic drug, and PS is psilocybin, a psilocin derivative or another related phosphorylated mushroom alkaloid.

In some specific examples this invention pertains to psilocin conjugates with formula 1a-i, or pharmaceutically acceptable salts, hydrates, or solvates thereof, wherein:

| compound | drugs | conjugates |
|---|---|---|
| 1a | CBD | 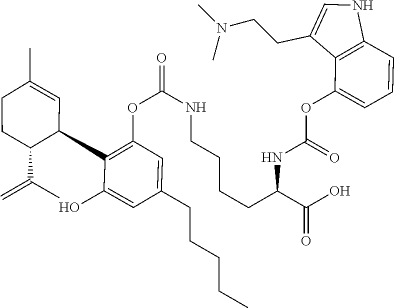 [SMILES: C=C(C)[C@@H]1CCC(C)=C[C@H]1c1c(O)cc(CCCCC)cc1OC(=O)NCCCC[C@@H](NC(=O)Oc1cccc2[nH]cc(CCN(C)C)c12)C(=O)O] |
| 1b | THC-V | 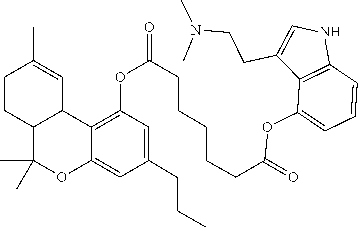 [SMILES: CCCc1cc(OC(=O)CCCCCC(=O)Oc2cccc3[nH]cc(CCN(C)C)c23)c2c(c1)OC(C)(C)C1CCC(C)=CC21] |
| 1c | Fluoxetine | 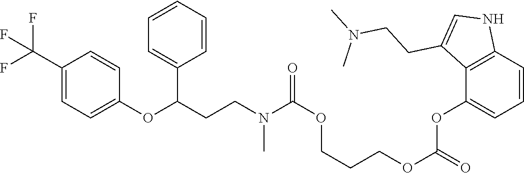 [SMILES: CN(C)CCc1c[nH]c2cccc(OC(=O)OCCCOC(=O)N(C)CCC(Oc3ccc(C(F)(F)F)cc3)c3ccccc3)c12] |
| 1d | Quetiapine | 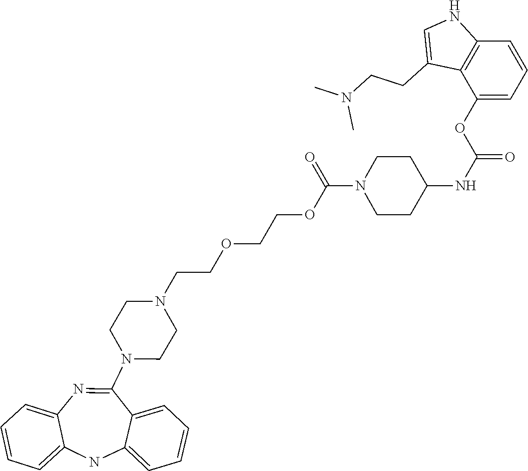 [SMILES: CN(C)CCc1c[nH]c2cccc(OC(=O)NC3CCN(C(=O)OCCOCCN4CCN(C5=Nc6ccccc6Nc6ccccc65)CC4)CC3)c12] |
| 1e | Paliperidone | 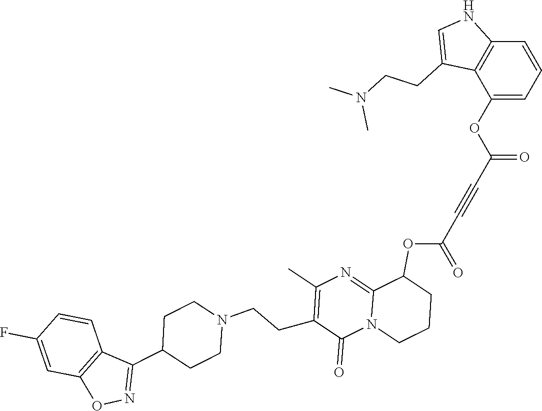 [SMILES: Cc1nc2n(c(=O)c1CCN1CCC(c3noc4cc(F)ccc34)CC1)CCCC2OC(=O)C#CC(=O)Oc1cccc2[nH]cc(CCN(C)C)c12] |
| 1f | Tapentadol | 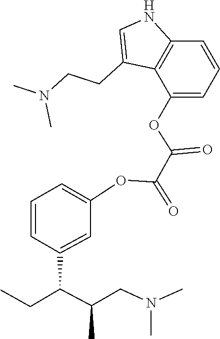 [SMILES: CC[C@H](c1cccc(OC(=O)C(=O)Oc2cccc3[nH]cc(CCN(C)C)c23)c1)[C@H](C)CN(C)C] |
| 1g | Pentazocine | 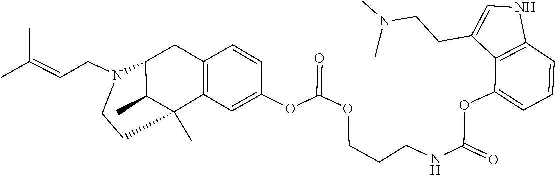 [SMILES: CC(C)=CCN1CC[C@@]2(C)c3cc(OC(=O)OCCCNC(=O)Oc4cccc5[nH]cc(CCN(C)C)c45)ccc3C[C@@H]1[C@@H]2C] |
| 1h | Galantamine | 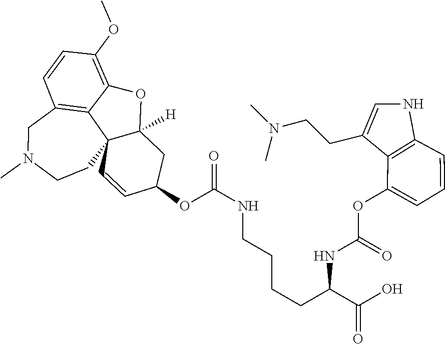 [SMILES: COc1ccc2c3c1O[C@H]1C[C@@H](OC(=O)NCCCC[C@@H](NC(=O)Oc4cccc5[nH]cc(CCN(C)C)c45)C(=O)O)C=C[C@@]31CCN(C)C2] |
| 1i | Pramipexole | 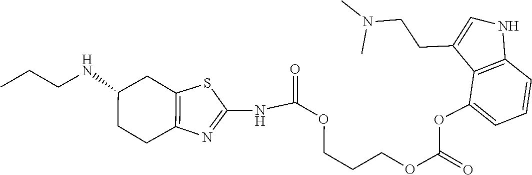 [SMILES: CCCN[C@H]1CCc2nc(NC(=O)OCCCOC(=O)Oc3cccc4[nH]cc(CCN(C)C)c34)sc2C1] |

Psilocin conjugates with formula 1a-i are expected to be released after administration to a subject, as illustrated in scheme 1:

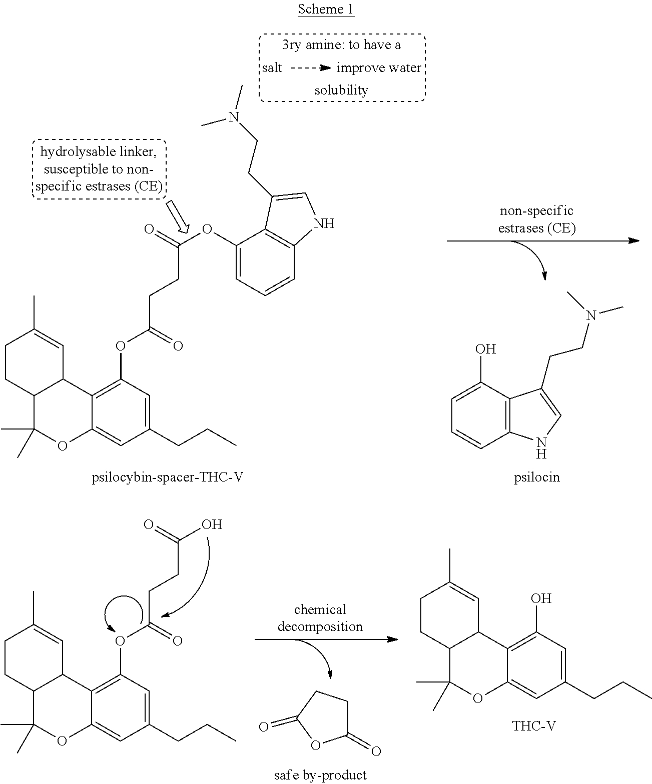

In some illustrative embodiments, this invention pertains to compounds having formula 2, or pharmaceutically acceptable salts, hydrates, or solvates thereof, wherein:

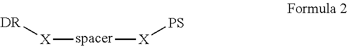

and wherein: DR is an additive or synergistic drug, and PS is psilocybin, a psilocin derivative or another related phosphorylated mushroom alkaloid.

In some specific examples this invention pertains to psilocin conjugates with formula 2a-i, or pharmaceutically acceptable salts, hydrates, or solvates thereof, wherein:

| compound | drugs | conjugates |
|---|---|---|
| 2a | CBD | 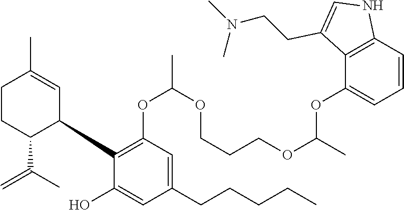 [SMILES: C=C(C)[C@@H]1CCC(C)=C[C@H]1c1c(O)cc(CCCCC)cc1OC(C)OCCCOC(C)Oc1cccc2[nH]cc(CCN(C)C)c12] |
| 2b | THC-V | 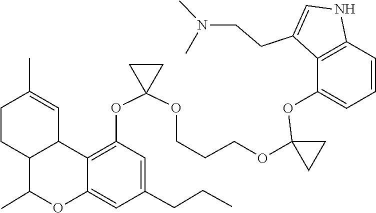 [SMILES: CCCc1cc2c(c(OC3(OCCCOC4(Oc5cccc6[nH]cc(CCN(C)C)c56)CC4)CC3)c1)C1C=C(C)CCC1C(C)O2] |
| 2c | Fluoxetine | 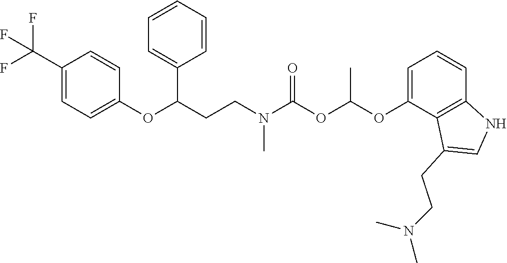 [SMILES: CC(OC(=O)N(C)CCC(Oc1ccc(C(F)(F)F)cc1)c1ccccc1)Oc1cccc2[nH]cc(CCN(C)C)c12] |
| 2d | Quetiapine | 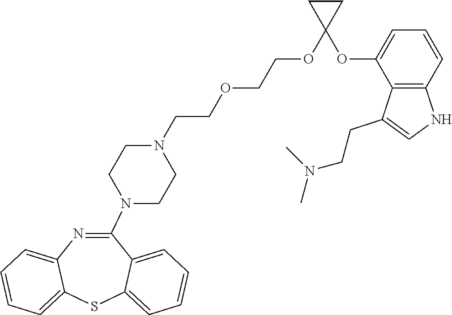 [SMILES: CN(C)CCc1c[nH]c2cccc(OC3(OCCOCCN4CCN(C5=Nc6ccccc6Sc6ccccc65)CC4)CC3)c12] |
| 2e | Paliperidone | 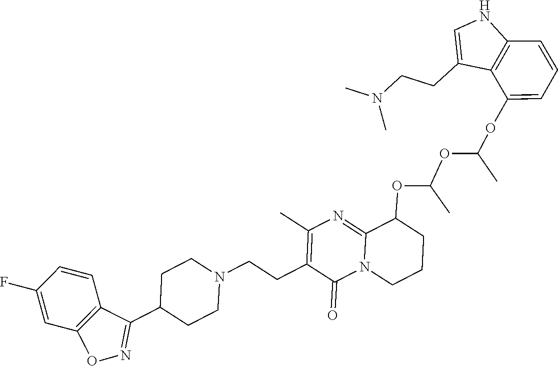 [SMILES: Cc1nc2n(c(=O)c1CCN1CCC(c3noc4cc(F)ccc34)CC1)CCCC2OC(C)OC(C)Oc1cccc2[nH]cc(CCN(C)C)c12] |
| 2f | Tapentadol | 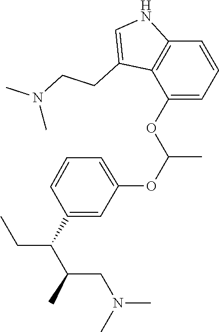 [SMILES: CC[C@H](c1cccc(OC(C)Oc2cccc3[nH]cc(CCN(C)C)c23)c1)[C@H](C)CN(C)C] |
| 2g | Pentazocine | 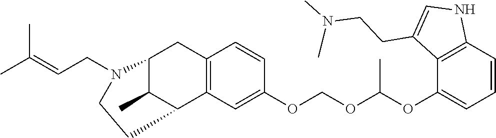 [SMILES: CC(C)=CCN1CC[C@H]2c3cc(OCOC(C)Oc4cccc5[nH]cc(CCN(C)C)c45)ccc3C[C@@H]1[C@@H]2C] |
| 2h | Galantamine | 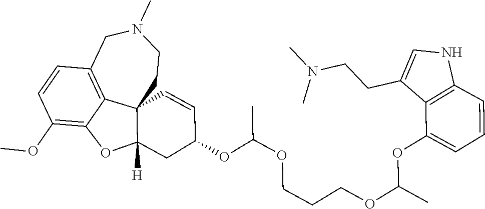 [SMILES: COc1ccc2c3c1O[C@H]1C[C@@H](OC(C)OCCCOC(C)Oc4cccc5[nH]cc(CCN(C)C)c45)C=C[C@@]31CCN(C)C2] |
| 2i | Pramipexole | 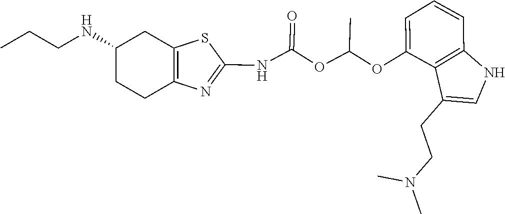 [SMILES: CCCN[C@H]1CCc2nc(NC(=O)OC(C)Oc3cccc4[nH]cc(CCN(C)C)c34)sc2C1] |

Psilocin conjugates with formula 2a-i are expected to be released after administration as illustrated in the following scheme 2:

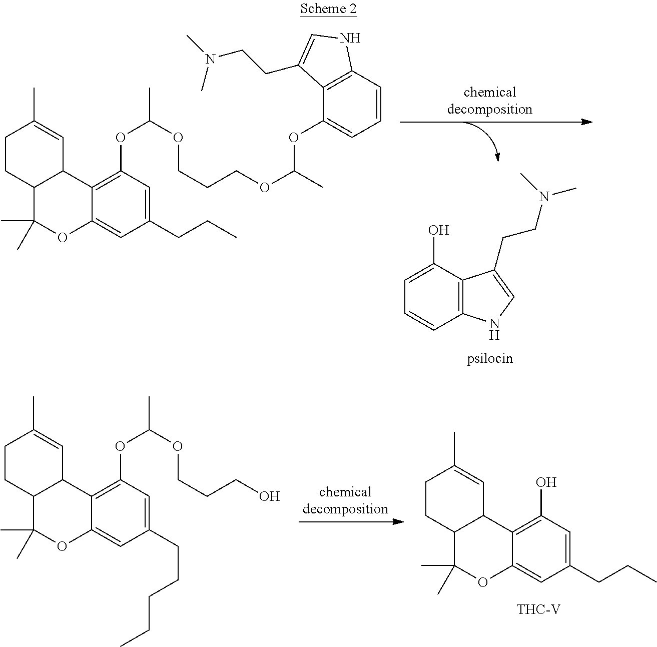

In some illustrative embodiments, this invention pertains to compounds having formula 3a-g, or pharmaceutically acceptable salts, hydrates, or solvates thereof, wherein:

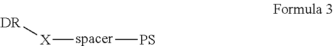

and wherein: DR is an additive or synergistic drug, and PS is psilocybin, a psilocin derivative or another phosphorylated mushroom alkaloid.

In some specific examples this invention pertains to psilocin conjugates with formula 3a-i, or pharmaceutically acceptable salts, hydrates, or solvates thereof, wherein:

| compound | drugs | conjugates |
|---|---|---|
| 3a | CBD | 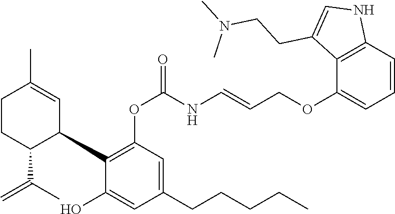 [SMILES: C=C(C)[C@@H]1CCC(C)=C[C@H]1c1c(O)cc(CCCCC)cc1OC(=O)N/C=C/COc1cccc2[nH]cc(CCN(C)C)c12] |
| 3b | THC-V | 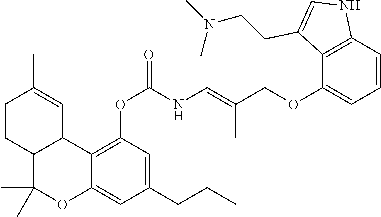 [SMILES: CCCc1cc(OC(=O)N/C=C(\C)COc2cccc3[nH]cc(CCN(C)C)c23)c2c(c1)OC(C)(C)C1CCC(C)=CC21] |
| 3c | Fluoxetine | 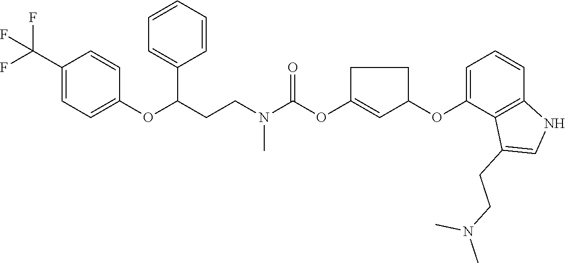 [SMILES: CN(C)CCc1c[nH]c2cccc(OC3C=C(OC(=O)N(C)CCC(Oc4ccc(C(F)(F)F)cc4)c4ccccc4)CC3)c12] |
| 3d | Quetiapine | 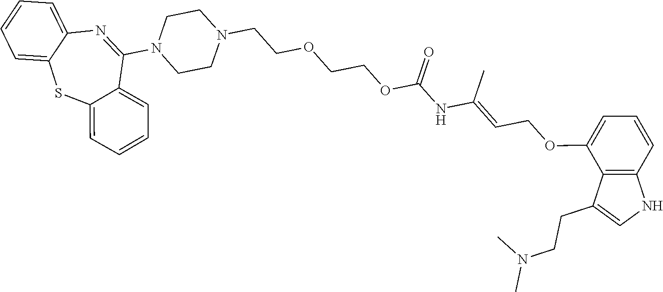 [SMILES: C/C(=C\COc1cccc2[nH]cc(CCN(C)C)c12)NC(=O)OCCOCCN1CCN(C2=Nc3ccccc3Sc3ccccc32)CC1] |
| 3e | Paliperidone | 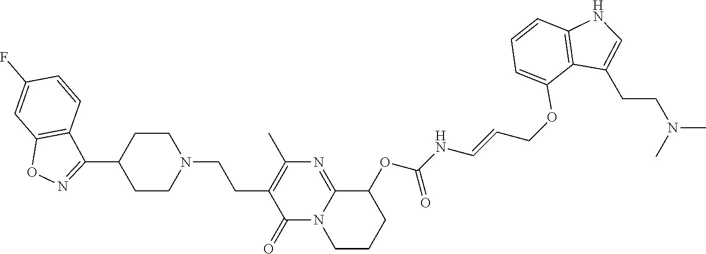 [SMILES: Cc1nc2n(c(=O)c1CCN1CCC(c3noc4cc(F)ccc34)CC1)CCCC2OC(=O)N/C=C/COc1cccc2[nH]cc(CCN(C)C)c12] |
| 3f | Tapentadol | 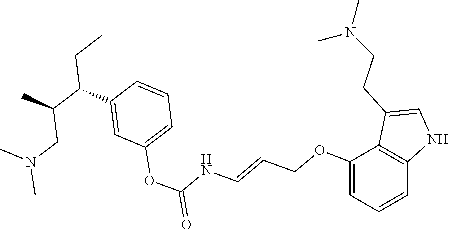 [SMILES: CC[C@H](c1cccc(OC(=O)N/C=C/COc2cccc3[nH]cc(CCN(C)C)c23)c1)[C@H](C)CN(C)C] |
| 3g | Pentazocine | 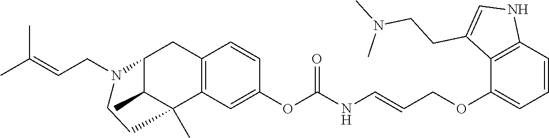 [SMILES: CC(C)=CCN1CC[C@@]2(C)c3cc(OC(=O)N/C=C/COc4cccc5[nH]cc(CCN(C)C)c45)ccc3C[C@@H]1[C@@H]2C] |
| 3h | Galantamine | 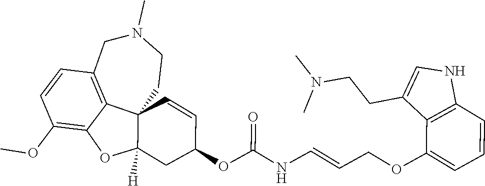 [SMILES: COc1ccc2c3c1O[C@@H]1C[C@H](OC(=O)N/C=C/COc4cccc5[nH]cc(CCN(C)C)c45)C=C[C@]31CCN(C)C2] |
| 3i | Pramipexole | 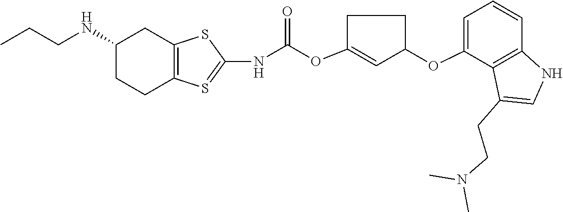 |

Psilocin conjugates with formula 3a-i are expected to be released after administration as illustrated in the following scheme 3:

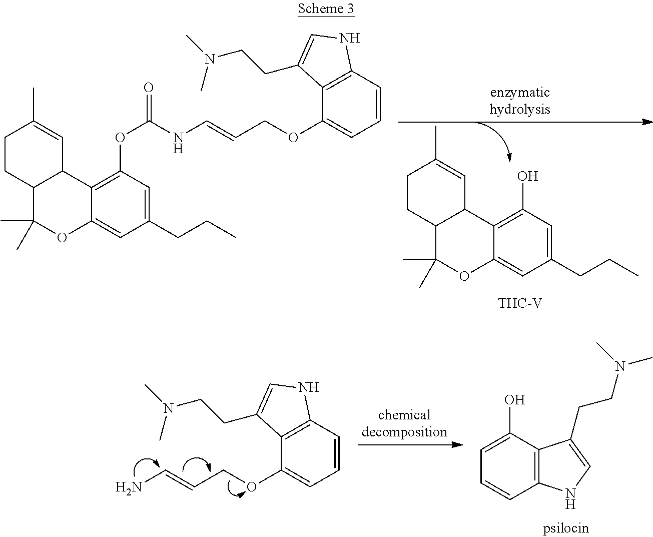

In some illustrative embodiments, this invention pertains to compounds having formula 4, or pharmaceutically acceptable salts, hydrates, or solvates thereof, wherein:

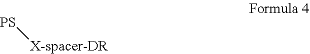

and wherein: DR is an additive or synergistic drug, and PS is psilocybin, a psilocin derivative or another phosphorylated mushroom alkaloid.

In some specific examples this invention pertains to psilocin conjugates with formula 4a-i, or pharmaceutically acceptable salts, hydrates, or solvates thereof, wherein:

| compound | drugs | conjugates |
|---|---|---|
| 4a | CBD | 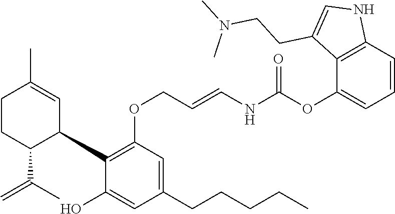 [SMILES: C=C(C)[C@@H]1CCC(C)=C[C@H]1c1c(O)cc(CCCCC)cc1OC/C=C/NC(=O)Oc1cccc2[nH]cc(CCN(C)C)c12] |
| 4b | THC-V | 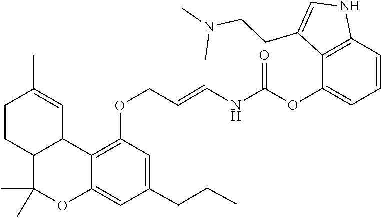 [SMILES: CCCc1cc(OC/C=C/NC(=O)Oc2cccc3[nH]cc(CCN(C)C)c23)c2c(c1)OC(C)(C)C1CCC(C)=CC21] |
| 4c | Fluoxetine | 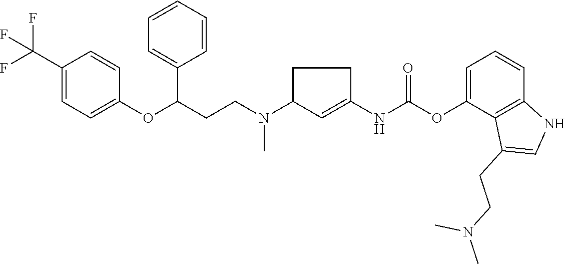 [SMILES: CN(C)CCc1c[nH]c2cccc(OC(=O)NC3=CC(N(C)CCC(Oc4ccc(C(F)(F)F)cc4)c4ccccc4)CC3)c12] |
| 4d | Quetiapine | 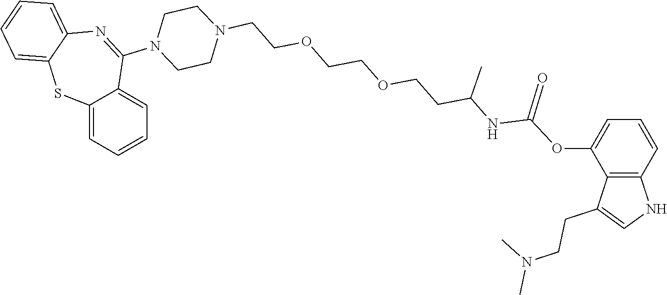 [SMILES: CC(CCOCCOCCN1CCN(C2=Nc3ccccc3Sc3ccccc32)CC1)NC(=O)Oc1cccc2[nH]cc(CCN(C)C)c12] |
| 4e | Paliperidone | 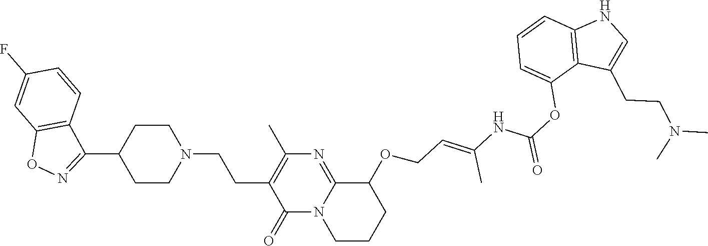 [SMILES: C/C(=C\COC1CCCn2c1nc(C)c(CCN1CCC(c3noc4cc(F)ccc34)CC1)c2=O)NC(=O)Oc1cccc2[nH]cc(CCN(C)C)c12] |
| 4f | Tapentadol | 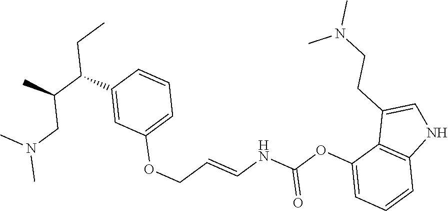 [SMILES: CC[C@H](c1cccc(OC/C=C/NC(=O)Oc2cccc3[nH]cc(CCN(C)C)c23)c1)[C@H](C)CN(C)C] |
| 4g | Pentazocine | 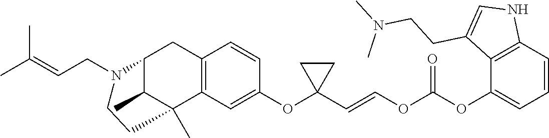 [SMILES: CC(C)=CCN1CC[C@@]2(C)c3cc(OC4(/C=C/OC(=O)Oc5cccc6[nH]cc(CCN(C)C)c56)CC4)ccc3C[C@@H]1[C@@H]2C] |
| 4h | Galantamine | 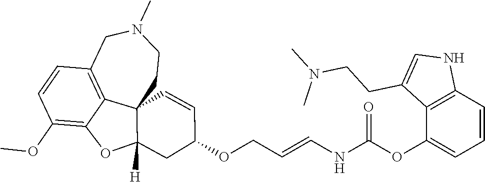 [SMILES: COc1ccc2c3c1O[C@H]1C[C@@H](OC/C=C/NC(=O)Oc4cccc5[nH]cc(CCN(C)C)c45)C=C[C@@]31CCN(C)C2] |
| 4i | Pramipexole | 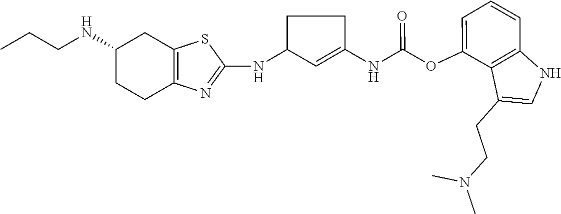 [SMILES: CCCN[C@H]1CCc2nc(NC3C=C(NC(=O)Oc4cccc5[nH]cc(CCN(C)C)c45)CC3)sc2C1] |

Psilocin conjugates with formula 4a-i are expected to be released after administration as illustrated in the following scheme 4:

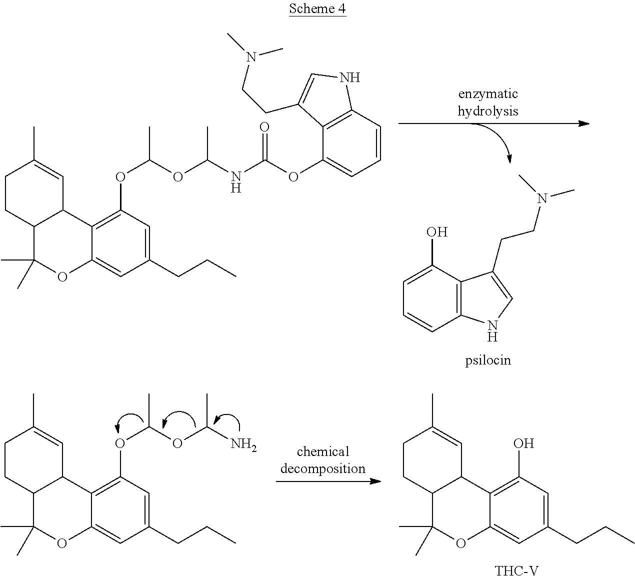

In some illustrative embodiments, this invention pertains to compounds having formula 5, in which the spacer is a therapeutic agent by itself, or pharmaceutically acceptable salts, hydrates, or solvates thereof, wherein:

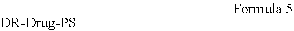

and wherein: DR is an additive or synergistic drug, and PS is psilocybin, a psilocin derivative or another phosphorylated mushroom alkaloid.

In some specific examples this invention pertains to psilocin conjugates with formula 5a-i, or pharmaceutically acceptable salts, hydrates, or solvates thereof, wherein:

| compound | drugs | conjugates |
|---|---|---|
| 5a | CBD-Gabapentin | 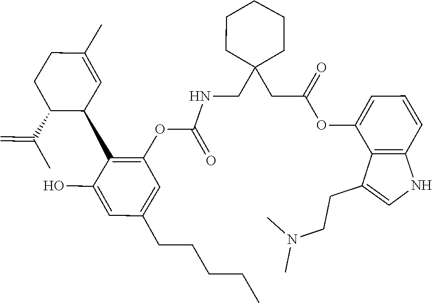 [SMILES: C=C(C)[C@@H]1CCC(C)=C[C@H]1c1c(O)cc(CCCCC)cc1OC(=O)NCC1(CC(=O)Oc2cccc3[nH]cc(CCN(C)C)c23)CCCCC1] |
| 5b | THC-V-Nalbuphine | 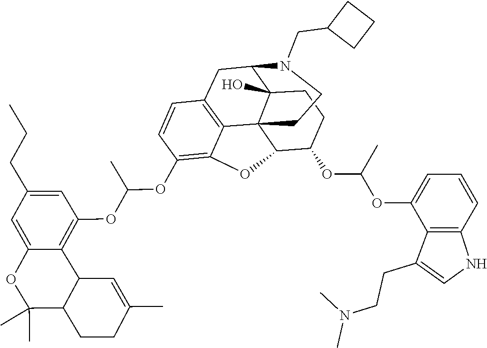 [SMILES: CCCc1cc(OC(C)Oc2ccc3c4c2O[C@H]2[C@@H](OC(C)Oc5cccc6[nH]cc(CCN(C)C)c56)CC[C@@]5(O)[C@@H](C3)N(CC3CCC3)CC[C@]425)c2c(c1)OC(C)(C)C1CCC(C)=CC21] |
| 5c | Fluoxetine-levodopa | 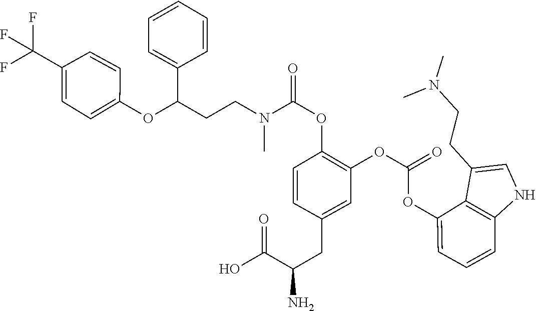 [SMILES: CN(C)CCc1c[nH]c2cccc(OC(=O)Oc3cc(C[C@@H](N)C(=O)O)ccc3OC(=O)N(C)CCC(Oc3ccc(C(F)(F)F)cc3)c3ccccc3)c12] |
| 5d | Quetiapine-baclofen | 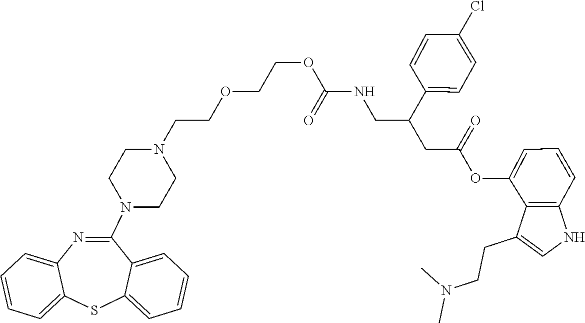 [SMILES: CN(C)CCc1c[nH]c2cccc(OC(=O)CC(CNC(=O)OCCOCCN3CCN(C4=Nc5ccccc5Sc5ccccc54)CC3)c3ccc(Cl)cc3)c12] |
| 5e | Paliperidone-levodopa | 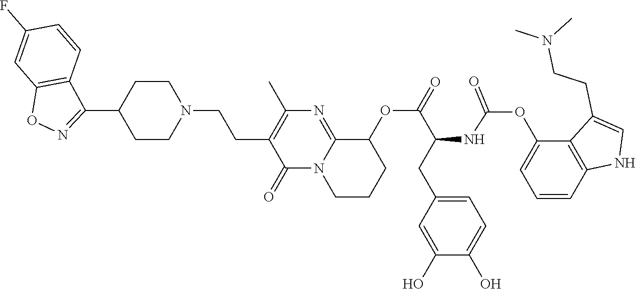 [SMILES: Cc1nc2n(c(=O)c1CCN1CCC(c3noc4cc(F)ccc34)CC1)CCCC2OC(=O)[C@H](Cc1ccc(O)c(O)c1)NC(=O)Oc1cccc2[nH]cc(CCN(C)C)c12] |
| 5f | Tapentadol-baclofen | 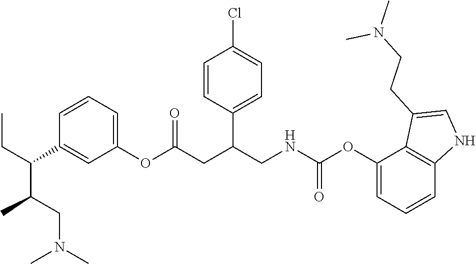 [SMILES: CC[C@H](c1cccc(OC(=O)CC(CNC(=O)Oc2cccc3[nH]cc(CCN(C)C)c23)c2ccc(Cl)cc2)c1)[C@H](C)CN(C)C] |
| 5g | Pentazocine-gabapentin | 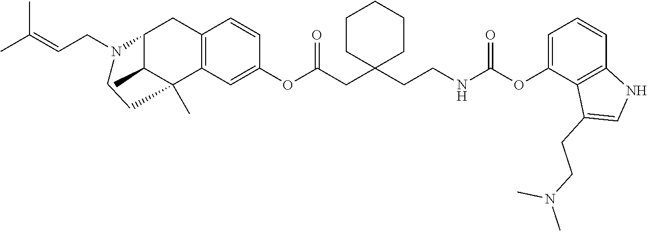 [SMILES: CC(C)=CCN1CC[C@@]2(C)c3cc(OC(=O)CC4(CCNC(=O)Oc5cccc6[nH]cc(CCN(C)C)c56)CCCCC4)ccc3C[C@@H]1[C@@H]2C] |
| 5h | Galantamine-Gabapentin | 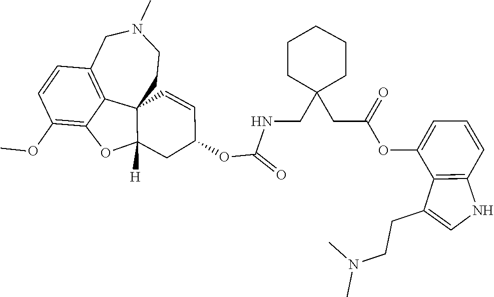 [SMILES: COc1ccc2c3c1O[C@H]1C[C@@H](OC(=O)NCC4(CC(=O)Oc5cccc6[nH]cc(CCN(C)C)c56)CCCCC4)C=C[C@@]31CCN(C)C2] |
| 5i | Carbidopa-levodopa | 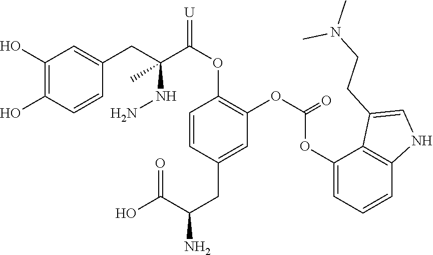 |

Psilocin conjugates with formula 5a-i are expected to be released after administration as illustrated in scheme 5:

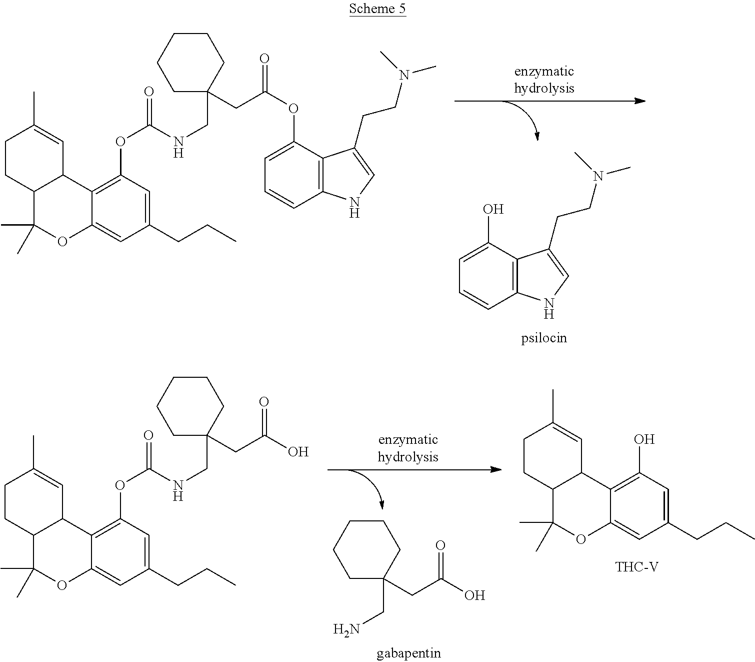

In some illustrative embodiments, this invention pertains to compounds having formula 6, or pharmaceutically acceptable salts, hydrates, or solvates thereof, wherein:

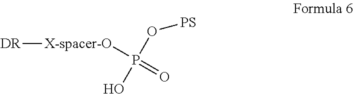

and wherein: DR is an additive or synergistic drug and PS is psilocybin, another psilocin derivative or another phosphorylated mushroom alkaloid.

In some specific examples this invention pertains to phosphorylated psilocin, or any other natural, synthetic, or semisynthetic derivatives, conjugates, in which the phosphate or its analogues or derivatives is (or are) part of the spacer or an “X” moiety with formula 6a-i, or both, or pharmaceutically acceptable salts, hydrates, or solvates thereof, wherein:

| compound | drugs | conjugates |
|---|---|---|
| 6a | CBD | 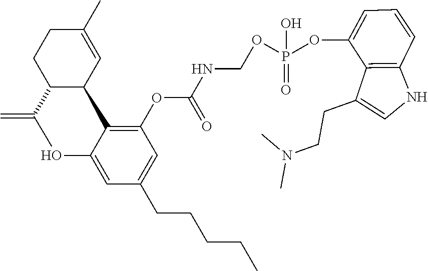 [SMILES: C=C1Oc2cc(CCCCC)cc(OC(=O)NCOP(=O)(O)Oc3cccc4[nH]cc(CCN(C)C)c34)c2[C@@H]2C=C(C)CC[C@@H]12] |
| 6b | THC-V | 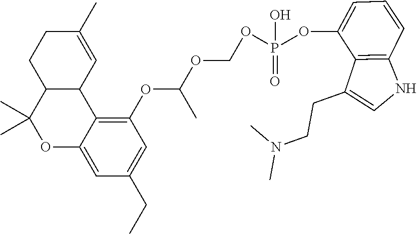 [SMILES: CCc1cc(OC(C)OCOP(=O)(O)Oc2cccc3[nH]cc(CCN(C)C)c23)c2c(c1)OC(C)(C)C1CCC(C)=CC21] |
| 6c | Fluoxetine | 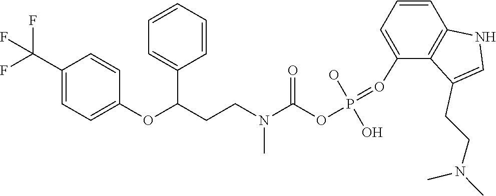 |
| 6d | Quetiapine | 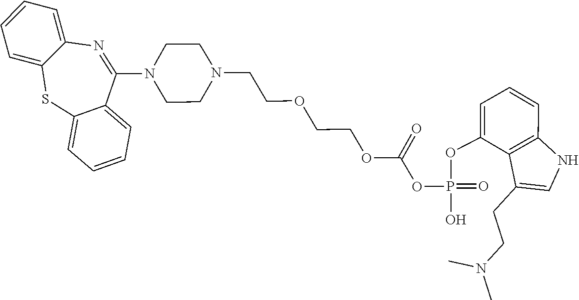 [SMILES: CN(C)CCc1c[nH]c2cccc(OP(=O)(O)OC(=O)OCCOCCN3CCN(C4=Nc5ccccc5Sc5ccccc54)CC3)c12] |
| 6e | Paliperidone | 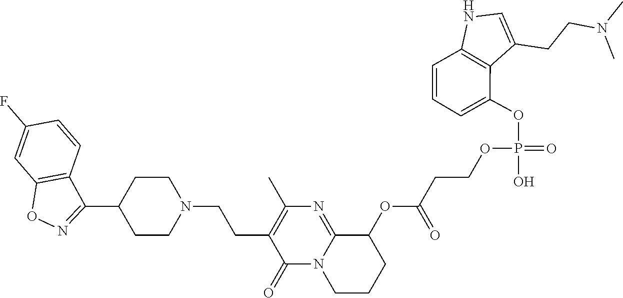 [SMILES: Cc1nc2n(c(=O)c1CCN1CCC(c3noc4cc(F)ccc34)CC1)CCCC2OC(=O)CCOP(=O)(O)Oc1cccc2[nH]cc(CCN(C)C)c12] |
| 6f | Tapentadol | 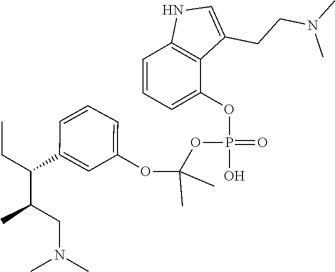 [SMILES: CC[C@H](c1cccc(OC(C)(C)OP(=O)(O)Oc2cccc3[nH]cc(CCN(C)C)c23)c1)[C@H](C)CN(C)C] |
| 6g | Pentazocine | 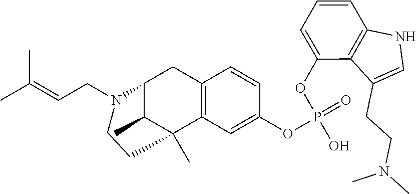 [SMILES: CC(C)=CCN1CC[C@@]2(C)c3cc(OP(=O)(O)Oc4cccc5[nH]cc(CCN(C)C)c45)ccc3C[C@@H]1[C@@H]2C] |
| 6h | Galantamine | 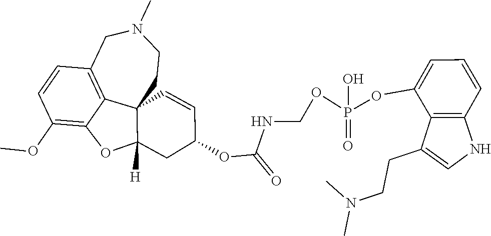 [SMILES: COc1ccc2c3c1O[C@H]1C[C@@H](OC(=O)NCOP(=O)(O)Oc4cccc5[nH]cc(CCN(C)C)c45)C=C[C@@]31CCN(C)C2] |
| 6i | Pramipexole | 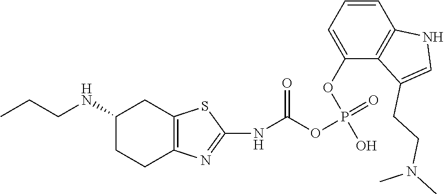 [SMILES: CCCN[C@H]1CCc2nc(NC(=O)OP(=O)(O)Oc3cccc4[nH]cc(CCN(C)C)c34)sc2C1] |

Phosphorylated conjugates with formula 6a-i are expected to be released after two sequential steps of enzymatic hydrolysis or by an alkaline phosphatase activation followed by hydrolysis of the chemically unstable bond according to schemes 6 and 7:

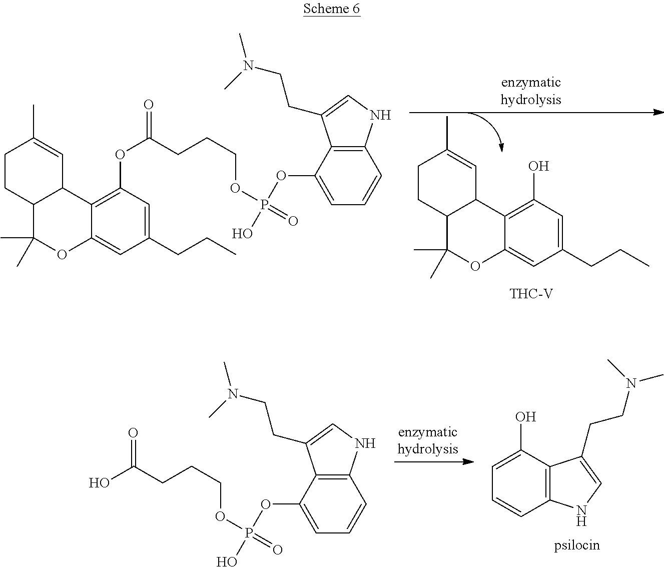

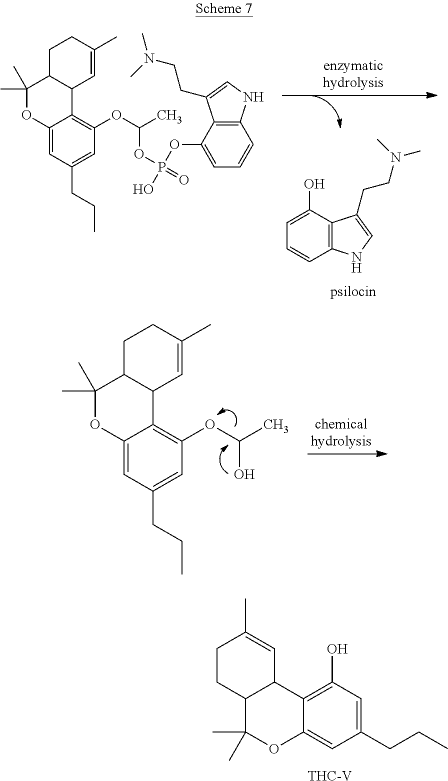

In some other embodiments, the addition of one or more halogen, in particular fluorine atom(s) on the carbon backbone of the spacer may be used to modulate the enzymatic and chemical stabilities, or to control sequence of hydrolysis as shown in specific examples of formula 7:

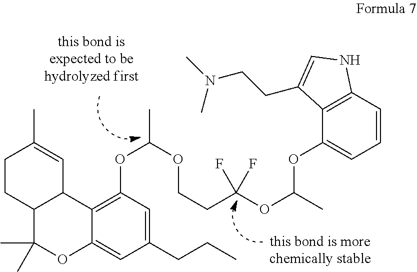 [SMILES: CCCc1cc(OC(C)OCCC(F)(F)OC(C)Oc2cccc3[nH]cc(CCN(C)C)c23)c2c(c1)OC(C)(C)C1CCC(C)=CC21]

In some other embodiments, addition of alkyl group(s), in particular methyl on the carbon backbone of the spacer may be used to modulate the enzymatic and chemical stabilities, to control the sequence of hydrolysis, or to reduce the toxicity as shown in specific examples of formula 8:

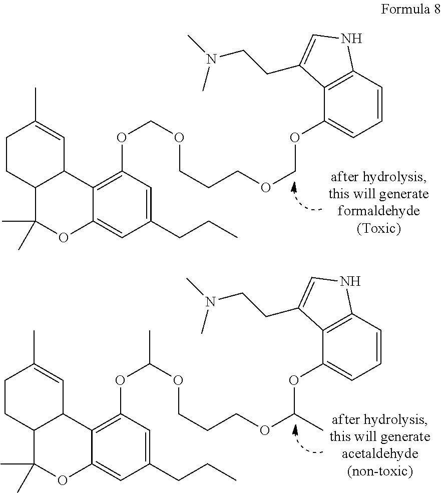

In another embodiment, PS is any psilocybin, psilocin derivative or another related phosphorylated mushroom alkaloid or metabolite, including, natural, synthetic or semisynthetic derivatives. Preferably, PS is psilocybin, psilocin, or other analogues such as baeocystin, norbaeocystin, bufotenine, and aeruginascin.

Preferable examples of natural phosphorylated mushroom alkaloids are:

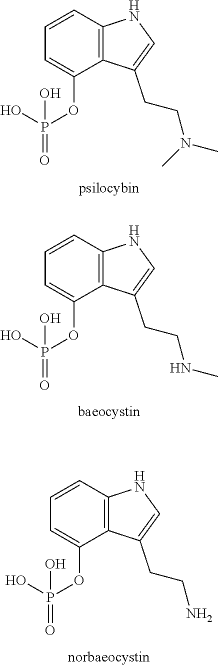 [SMILES: CN(C)CCc1c[nH]c2cccc(OP(=O)(O)O)c12 ; CNCCc1c[nH]c2cccc(OP(=O)(O)O)c12 ; NCCc1c[nH]c2cccc(OP(=O)(O)O)c12]

Preferable examples of synthetic or semisynthetic phosphorylated related mushrooms alkaloids are:

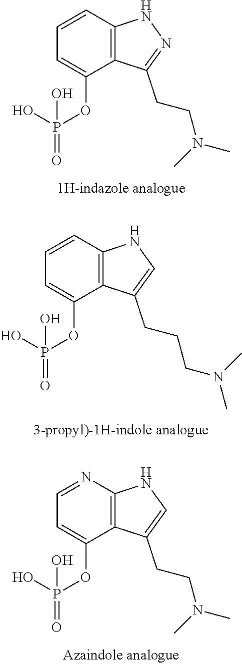 [SMILES: CN(C)CCc1n[nH]c2cccc(OP(=O)(O)O)c12 ; CN(C)CCCc1c[nH]c2cccc(OP(=O)(O)O)c12 ; CN(C)CCc1c[nH]c2nccc(OP(=O)(O)O)c12]

In another embodiment, DR is an additive or synergistic drug or its metabolite selected from pregabalin, gabapentin, topiramate, morphine, levodopa, selective serotonin reuptake inhibitor (SSRI) (e.g., citalopram), amisulpride, lurasidone, paliperidone, paliperidone palmitate, risperidone, ziprasidone, perospirone, doxorubicin, melperone, aripiprazole, brexpiprazole, cariprazine, olanzapine, quetiapine, flouxetine, calcitonin, pseudoephedrine, piracetam, levetiracetam, sitagliptin, silodosin, hydrochlorothiazide, ezetimibe, propranolol, atenolol, nadolol, pindolol, sotalol, timolol, penbutolol, oxprenolol, carvedilol, carteolol, bucindolol, acebutanol, betaxolol, esmolol, nebivolol, bisoprolol, celiprolol, metoprolol, azelnidipine, barnidipine, manidipine, lercandipine, efonidipine, benidipine, brimonidine, bortezomib, ledipasvir, daclatasvir, ombitasvir, elbasvir, lamivudine, dopamine, 5-hydroxytryptamine, levodopa, pramipexole, ropinirole, rotigotine, apomorphine, tacrine, rivastigmine, donepezil, galantamine, vigabatrin, lamotrigine, tiagabine, amitriptyline, nortriptyline and histamine or cannabinoids. Preferably, DR is a cannabinoid.

In another embodiment, DR is one or more cannabinoid derivatives or metabolites, including, synthetic, or semisynthetic derivatives. Preferably the one or more cannabinoids are taken from the group: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures and salts, and mixtures and combinations thereof. Preferably, DR is CBD or THC-V.

In some illustrative embodiments, X is linear, cyclic, or branched hydrocarbon chain, with or without heteroatom(s), oxygen, sulfur, with any oxidation status, NH or substituted nitrogen. In other illustrative embodiments, preferably, X is a phosphate, phosphonate, or a substituted phosphate or phosphonate.

In some illustrative embodiments, Spacer is a linear, branched or cyclic alkane, alkene or alkyne, optionally halo substituted, amino acid, acetal, ketal, peptide, aryl, heteroaryl, or heterocycloalkyl each of which is optionally substituted. In some illustrative embodiments, any of Spacer, Spacer-X, or X-Spacer-X may be a hydrolysable peptide or protein.

In any of the embodiments described above in which two or more cannabinoid components can be attached, each cannabinoid component can be the same or different, and, when spacers are used, each spacer can be the same or different.

It is appreciated herein that the compounds described herein may be used alone or in combination with other psychiatric therapeutic(s) that may be therapeutically effective by the same or different modes of action. In addition, it is appreciated herein that the compounds described herein may be used in combination with other therapeutics that are administered to treat other symptoms of psychiatric diseases, such as compounds administered to relieve pain, allergy, swelling nausea/vomiting, and the like.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 1%, within 5%, or within 10% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

As used herein, the term “alkyl” refers to a saturated monovalent chain of carbon atoms, which may be optionally branched. It is understood that in embodiments that include alkyl, illustrative variations of those embodiments include lower alkyl, such as C₁ to C₉ alkyl, methyl, ethyl, propyl, 3-methylbutyl, and the like.

As used herein, the terms “cyclic alkane” refers to a monovalent chain of carbon atoms, a portion of which forms a ring. It is understood that in embodiments that include a cyclic alkane moiety, illustrative variations of those embodiments include a lower cyclic alkane moiety, such as C₃-C₆ cycloalkyl, cyclopropyl, cyclobutyl, 3-methylcyclohexyl, and the like.

As used herein, the term “cyclic alkene” refers to an unsaturated monovalent chain of carbon atoms, a portion of which forms a ring. It is understood that in embodiments that include a cyclic alkene moiety, illustrative variations of those embodiments include a lower cyclic alkene moiety, such as C₃-C₆ cycloalkenyl, cyclopentenyl, cyclohexenyl, and the like.

As used herein, the terms “aryl” used alone or as part of a phrase such as “aralkyl” or “alkylaryl” refer to monocyclic, bicyclic or fused ring systems, with at least one aromatic ring, having 5- to 12 ring members, wherein at least one ring in the system is aromatic and wherein each ring in the system contains 3 to 7 ring members. The term “aryl” may be used interchangeably with the term “aryl ring”. Examples of aryl rings include phenyl, biphenyl, naphthyl, anthracyl and the like, which may bear one or more substituents.

As used herein, the term “heteroaryl” refers 4-, 5-, 6-, or 7-membered ring having 1 to 4 heteroatom selected from O, N, and S, or 8-, 9-, or 10-membered ring having 1 to 6 heteroatoms selected from O, N, and S, or a salt thereof. Examples of heteroaryl groups include, pyridine, pyrimidine, pyridazine, quinazoline, quinoline, indole, pyrole, pyrazole, imidazole, furan, benzofuran, thiophene, and benzothiophene.

As used herein, the term “hetero-atom” refers to non-carbon and non-hydrogen atoms such as N, O, S, Se, P, and the like, preferably N, O or S atoms.

As used herein, the term “sulfer oxidation status” refers to sulfide, sulfone, sulfoxide, and sulfonamide.

As used herein, “heterocycloalkyl,” by itself or in combination with another term, refers to a saturated monovalent ring of carbon atoms, consisting of the stated number of carbon atoms, where one or more carbon atom(s), such as 1, 2, 3 or 4 carbon atom(s), and the associated hydrogen atom(s) have been independently replaced with the same or different heteroatoms, for example, nitrogen, oxygen or sulfur. The carbon atom(s) being replace may be at any position of the ring. Examples of heterocycloalkyl groups include tetrahydro-2H-pyran, tetrahydro-2H-thiopyran, —NH-alkyl, -alkylene-O-alkyl, and the like.

It is understood that each of alkyl, cycloalkane, alkene, and cycloalkene moieties may be optionally substituted with independently selected groups such as halide, alkyl, alkoxy, hydroxy, hydroxyalkyl, carboxylic acid and derivatives thereof, including esters, nitrile, amides, and nitrites, acyloxy, aminoalkyl and dialkylamino, acylamino, thio, and the like, and combinations thereof.

The term “halide” refers to fluoride, chloride, bromide, or iodide.

The term “optionally substituted,” or “optional substituents,” as used herein, means that the groups in question are either unsubstituted or substituted with one or more of the substituents specified. When the groups in question are substituted with more than one substituent, the substituents may be the same or different. Moreover, when using the terms “independently,” means that the groups in question may be the same or different. Certain of the herein defined terms may occur more than once in the structure, and upon such occurrence each term shall be defined independently of the other.

The term “patient” includes human and non-human animals such as companion animals such as dogs and cats and the like, and livestock animals. Livestock animals are animals raised for food production. The patient to be treated is preferably a mammal, in particular a human being.

The terms “pharmaceutically acceptable diluent” or “pharmaceutically acceptable excipient” are art—recognized and refer to a pharmaceutically acceptable material, composition or vehicle, such as a liquid or solid filler, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose and maltose: (2) starches, such as corn starch and gelatinized starch: (3) cellulose, and its derivatives, such as carboxymethyl cellulose salt, and hydroxypropylmethyl cellulose: (4) thickening agents such as gelatin and tragacanth; (5) disintegrants such as copovidone: (6) other excipients, such as cocoa butter and suppository waves and pyrogen—free water for sterile products: and (7) other non-toxic compatible substances employed in pharmaceutical formulations.

The term “subject” in the present disclosure refers to human patients but is not limited to humans and may include animals.

As used herein, the term “administering” includes all means of introducing the compounds and compositions described herein to the patient, including, but are not limited to, topical, oral, intravenous, intramuscular, transdermal, inhalation, buccal, ocular, vaginal, rectal, and the like. The compounds and compositions described herein may be administered in unit dosage forms or formulations containing conventional nontoxic pharmaceutically acceptable carriers, adjuvants, and vehicles.

In some other embodiments, this invention pertains to a pharmaceutical composition comprising a compound disclosed herein, or a pharmaceutically acceptable salt thereof, together with one or more pharmaceutically acceptable diluents, and excipients.

In some embodiments, this invention pertains to a pharmaceutical composition comprising a compound disclosed herein, in combination with one or more other therapeutically active compounds by the same or different mode of action, and one or more pharmaceutically acceptable diluents, and excipients.

In some other embodiments, psilocybin and psilocin exerts its pharmacological effect via modulation of serotonin and dopaminergic receptors including 5HT_{2B}, 5HT_{1D}, 5HT_{1E}, 5HT_{1A}, 5HT_{5A}, 5HT₇, 5HT₆, 5HT_{2C}, 5HT_{1B}, 5HT_{2A}, D1, D3; while the DR moiety are modulators of cannabinoid receptor CB1, CB2 and other receptors.

In some embodiments, modulators pertain to allosteric modulators, agonist, biased agonist, antagonist, biased antagonist or partial agonist of any opiate receptor(s), blocking the reuptake of serotonin, modulating the level of neurotransmitters in CNS or peripheral tissues, modulating the level of cellular secondary messengers or modulating the phosphorylated level of cellular enzymes or proteins. Several studies support the synergistic action of the dual modulating of CB1 and 5HT_{2A} receptors in terms of blocking psychotropic/hallucination adverse effects.

In some embodiments, this invention pertains to a method for treating a patient of psychological disorder(s), the method comprising the step of administering a therapeutically effective amount of a compound disclosed herein, together with one or more pharmaceutically acceptable diluents, and excipients, to the patient in need of relief from said psychological disorder(s).

### Example 1: Synthesis of Psilocin-THC-V Conjugate

The t-Bu-protected dicarboxylic acid (1 equiv.) is activated using 1,1′-carbonyldiimidazole (CDI) (1 equiv.), and then the psilocin is added. The reaction mixture is stirred at 80° C. for 12 hours. Progress of reaction may be monitored by TLC. After complete conversion, the reaction is quenched with distilled water (50 mL), organic material is extracted with ethyl acetate (50 mL×3), collected, dried over anhydrous MgSO₄, and concentrated under reduced pressure. The crude intermediate is dissolved in degassed absolute ethanol (20 mL), charged with Pd/C (10%, 20 mg), hydrogen gas is applied, and the reaction is stirred at room temperature for 8 hours. After completion of the reaction, as may be monitored by TLC, the reaction mixture is filtered through Celite 545, and the solvent is removed under vacuum. The free acid is then purified by partitioning between aqueous solution of sodium carbonate (1M, 50 mL), and DCM (25 mL). The aqueous layer is separated and acidified with ammonium hydrochloride to neutral pH. The precipitate is collected, washed thoroughly with distilled water and dried. The obtained solid material (1 equiv.) is then dissolved in dry DMF (10 mL), CDI (1 equiv.) is added, and the reaction mixture is stirred at room temperature for 2 hours. After that, THC-V (1 equiv.) is added, and the temperature is raised to 80° C. After completion or the reaction, 50 mL of cooled distilled water is added to the reaction mixture and solid flocculates are collected by filtration and purified by normal phase column chromatography using hexane:ethyl acetate (4:1) (scheme 8).

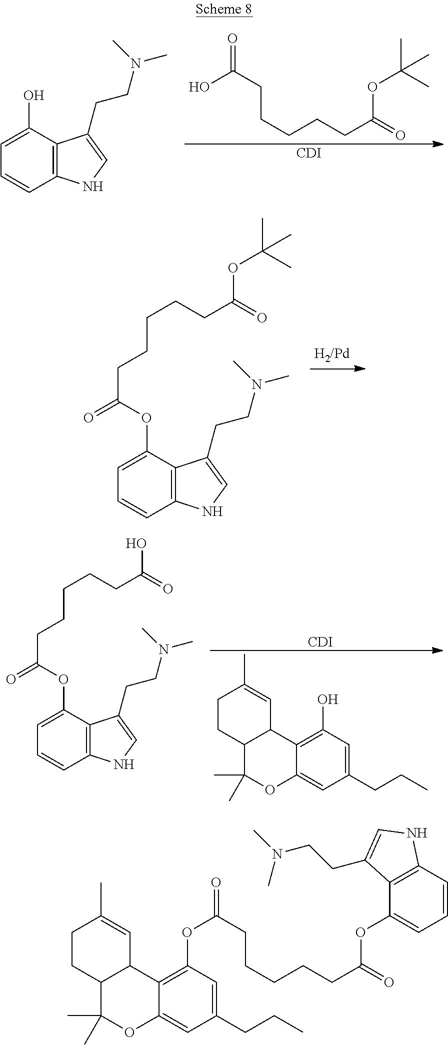

Expansion of synthetic protocol. The first discussed synthetic pathway (Scheme 8) is highly flexible and can be modified easily to cover all compounds belong to formula 1-5. As an illustrative example, Scheme 9 was designed to synthesize THC—V-Gabapentin-Psilocin.

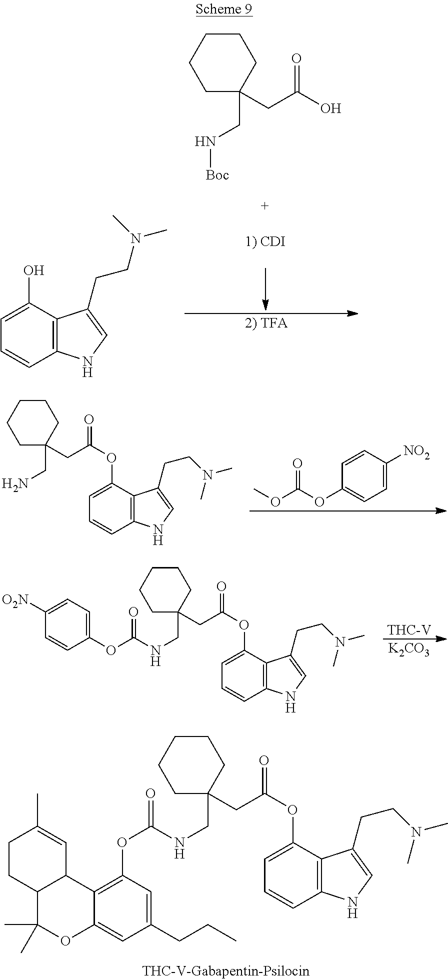

In another embodiment, the 1,1′-carbonyldiimidazole (CDI) can be replaced by other coupling reagents including: phosgene, trichloroacetyl chloride, 1,1′-carbonylbis(2-methylimidazole), N,N′-disuccinimidyl carbonate, 4-nitrophenylchloroformate, and bis(4-nitrophenyl)carbonate, bis(pentafluorophenyl)carbonate.

In another embodiment, synthesis of a carbamate moiety (as in Scheme 9) can be achieved by selecting reagents, including: phosgene, trichloroacetyl chloride, 1,1′-carbonyldiimidazole (CDI), 1,1′-carbonylbis(2-methylimidazole), N,N′-disuccinimidyl carbonate, 4-nitrophenylchloroformate, bis(4-nitrophenyl)carbonate, bis(pentafluorophenyl)carbonate, and then, optionally, treated with the second partner.

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A compound, or pharmaceutically acceptable salt thereof, selected from the group consisting of formulas 1 to 6: 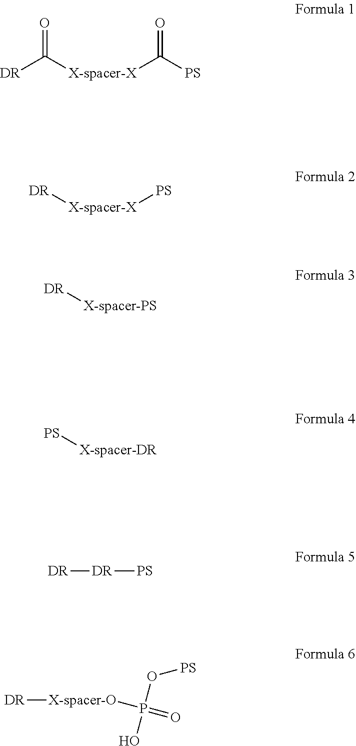
wherein:
PS is psilocybin, a psilocin derivative, or a relate phosphorylated mushroom alkaloid;
each DR is, independently, an additive or synergistic compound;
each X is, independently, a linear, cyclic or branched alkyl carbon chain, with or without heteroatom(s), phosphate, phosphonate, substituted, phosphate or phosphonate, oxygen, sulfur with any oxidation status, NH, or substituted nitrogen; and
SPACER is a substituted or unsubstituted linear, branched, or cyclic alkane, alkene, or alkyne, amino acid, acetal, ketal, peptide, aryl, heteroaryl, or heterocycloalkyl group.

2. The compound of claim 1, wherein the compound is a compound of formula 1; PS is psilocybin; and DR is selected from the group consisting of: CBD, THC-V, fluoxetine, quetiapine, paliperidone, tapentadol, pentazocine, galantamine, and pramipexole.

3. The compound of claim 2, wherein the compound is a compound of formula 1a.

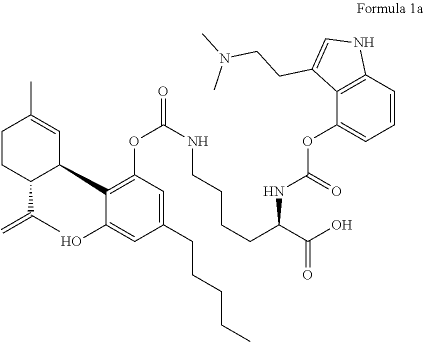 [SMILES: C=C(C)[C@@H]1CCC(C)=C[C@H]1c1c(O)cc(CCCCC)cc1OC(=O)NCCCC[C@@H](NC(=O)Oc1cccc2[nH]cc(CCN(C)C)c12)C(=O)O]

4. The compound of claim 2, wherein the compound is a compound of formula 1b.

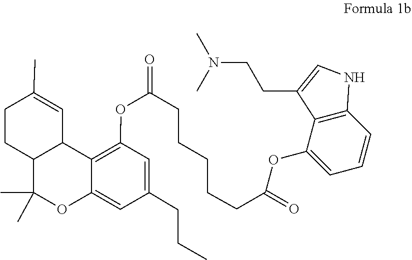 [SMILES: CCCc1cc(OC(=O)CCCCCC(=O)Oc2cccc3[nH]cc(CCN(C)C)c23)c2c(c1)OC(C)(C)C1CCC(C)=CC21]

5. The compound of claim 2, wherein the compound is a compound of formula 1c.

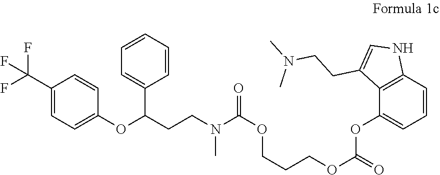 [SMILES: CN(C)CCc1c[nH]c2cccc(OC(=O)OCCCOC(=O)N(C)CCC(Oc3ccc(C(F)(F)F)cc3)c3ccccc3)c12]

6. The compound of claim 2, wherein the compound is a compound of formula 1d.

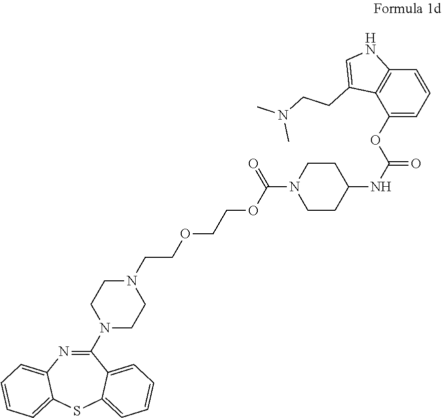 [SMILES: CN(C)CCc1c[nH]c2cccc(OC(=O)NC3CCN(C(=O)OCCOCCN4CCN(C5=Nc6ccccc6Sc6ccccc65)CC4)CC3)c12]

7. The compound of claim 2, wherein the compound is a compound of formula 1e.

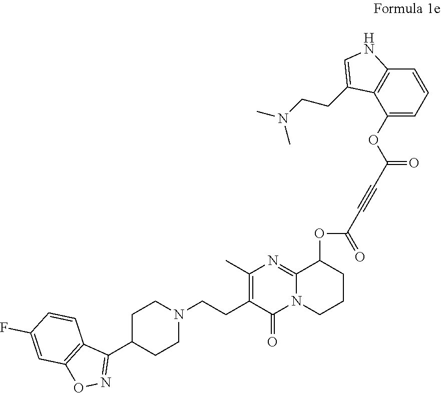 [SMILES: Cc1nc2n(c(=O)c1CCN1CCC(c3noc4cc(F)ccc34)CC1)CCCC2OC(=O)C#CC(=O)Oc1cccc2[nH]cc(CCN(C)C)c12]

8. The compound of claim 2, wherein the compound is a compound of formula 1f.

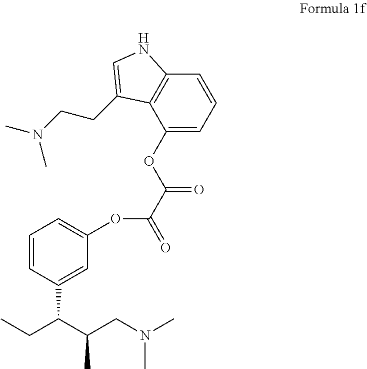 [SMILES: CC[C@H](c1cccc(OC(=O)C(=O)Oc2cccc3[nH]cc(CCN(C)C)c23)c1)[C@H](C)CN(C)C]

9. The compound of claim 2, wherein the compound is a compound of formula 1g

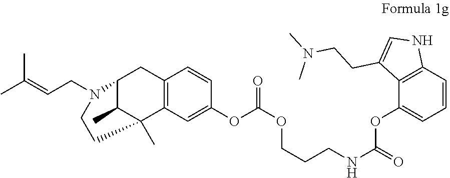 [SMILES: CC(C)=CCN1CC[C@@]2(C)c3cc(OC(=O)OCCCNC(=O)Oc4cccc5[nH]cc(CCN(C)C)c45)ccc3C[C@@H]1[C@@H]2C]

10. The compound of claim 2, wherein the compound is a compound of formula 1h.

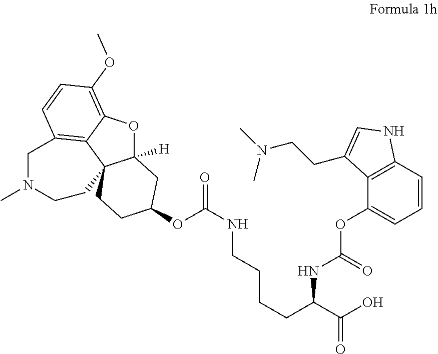 [SMILES: COc1ccc2c3c1O[C@H]1C[C@@H](OC(=O)NCCCC[C@@H](NC(=O)Oc4cccc5[nH]cc(CCN(C)C)c45)C(=O)O)CC[C@@]31CCN(C)C2]

11. The compound of claim 2, wherein the compound is a compound of formula 1i.

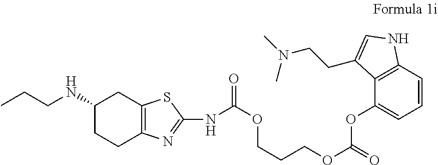 [SMILES: CCCN[C@H]1CCc2nc(NC(=O)OCCCOC(=O)Oc3cccc4[nH]cc(CCN(C)C)c34)sc2C1]

12. The compound of claim 1, wherein the compound is a compound of formula 2; PS is psilocybin; and DR is selected from the group consisting of: CBD, THC-V, fluoxetine, quetiapine, paliperidone, tapentadol, pentazocine, galantamine, and pramipexole.

13. The compound of claim 12, wherein the compound is a compound of formula 2a.

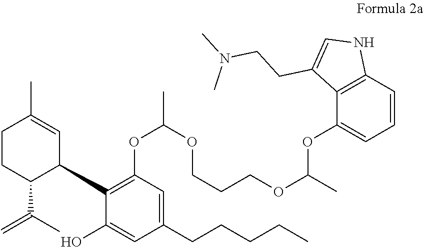 [SMILES: C=C(C)[C@@H]1CCC(C)=C[C@H]1c1c(O)cc(CCCCC)cc1OC(C)OCCCOC(C)Oc1cccc2[nH]cc(CCN(C)C)c12]

14. The compound of claim 12, wherein the compound is a compound of formula 2b.

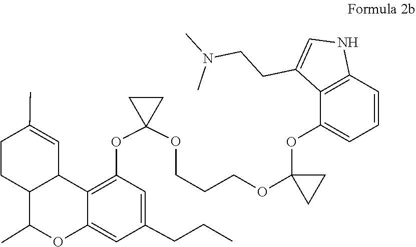 [SMILES: CCCc1cc2c(c(OC3(OCCCOC4(Oc5cccc6[nH]cc(CCN(C)C)c56)CC4)CC3)c1)C1C=C(C)CCC1C(C)O2]

15. The compound of claim 12, wherein the compound is a compound of formula 2c.

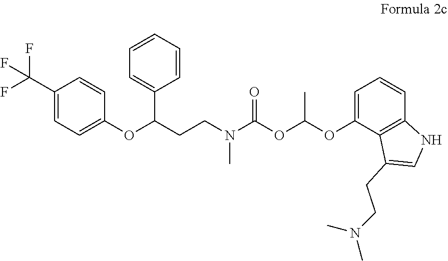 [SMILES: CC(OC(=O)N(C)CCC(Oc1ccc(C(F)(F)F)cc1)c1ccccc1)Oc1cccc2[nH]cc(CCN(C)C)c12]

16. The compound of claim 12, wherein the compound is a compound of formula 2d.

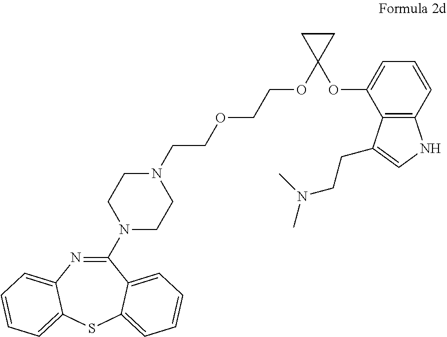 [SMILES: CN(C)CCc1c[nH]c2cccc(OC3(OCCOCCN4CCN(C5=Nc6ccccc6Sc6ccccc65)CC4)CC3)c12]

17. The compound of claim 12, wherein the compound is a compound of formula 2e.

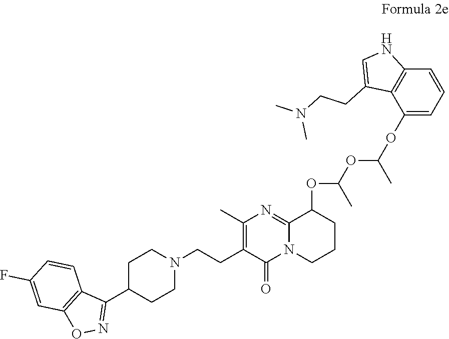 [SMILES: Cc1nc2n(c(=O)c1CCN1CCC(c3noc4cc(F)ccc34)CC1)CCCC2OC(C)OC(C)Oc1cccc2[nH]cc(CCN(C)C)c12]

18. The compound of claim 12, wherein the compound is a compound of formula 2f.

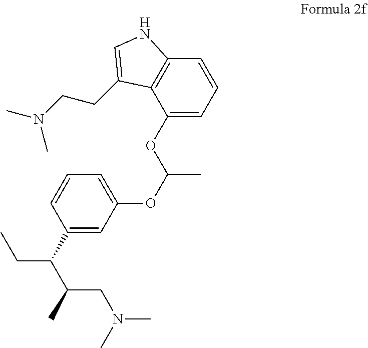 [SMILES: CC[C@H](c1cccc(OC(C)Oc2cccc3[nH]cc(CCN(C)C)c23)c1)[C@H](C)CN(C)C]

19. The compound of claim 12, wherein the compound is a compound of formula 2g.

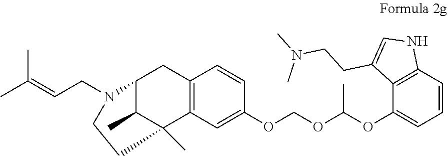 [SMILES: CC(C)=CCN1CC[C@@]2(C)c3cc(OCOC(C)Oc4cccc5[nH]cc(CCN(C)C)c45)ccc3C[C@@H]1[C@@H]2C]

20. The compound of claim 12, wherein the compound is a compound of formula 2h.

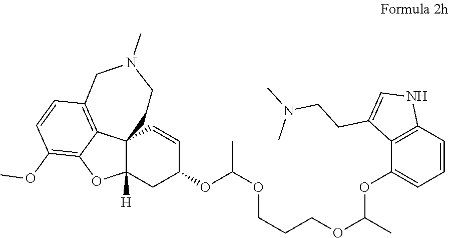 [SMILES: COc1ccc2c3c1O[C@H]1C[C@@H](OC(C)OCCCOC(C)Oc4cccc5[nH]cc(CCN(C)C)c45)C=C[C@@]31CCN(C)C2]

21. The compound of claim 12, wherein the compound is a compound of formula 2i.

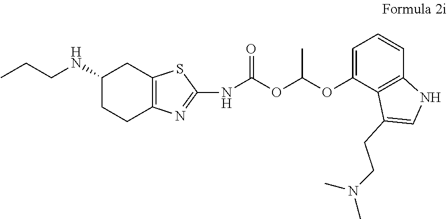 [SMILES: CCCN[C@H]1CCc2nc(NC(=O)OC(C)Oc3cccc4[nH]cc(CCN(C)C)c34)sc2C1]

22. The compound of claim 1, wherein the compound is a compound of formula 3; PS is psilocybin; and DR is selected from the group consisting of: CBD, THC-V, fluoxetine, quetiapine, paliperidone, tapentadol, pentazocine, galantamine, and pramipexole.

23. The compound of claim 22, wherein the compound is a compound of formula 3a.

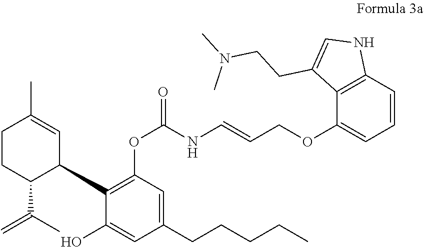 [SMILES: C=C(C)[C@@H]1CCC(C)=C[C@H]1c1c(O)cc(CCCCC)cc1OC(=O)N/C=C/COc1cccc2[nH]cc(CCN(C)C)c12]

24. The compound of claim 22, wherein the compound is a compound of formula 3b.

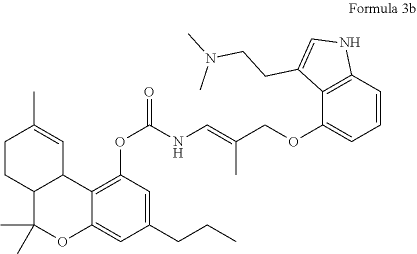 [SMILES: CCCc1cc(OC(=O)N/C=C(\C)COc2cccc3[nH]cc(CCN(C)C)c23)c2c(c1)OC(C)(C)C1CCC(C)=CC21]

25. The compound of claim 22, wherein the compound is a compound of formula 3c.

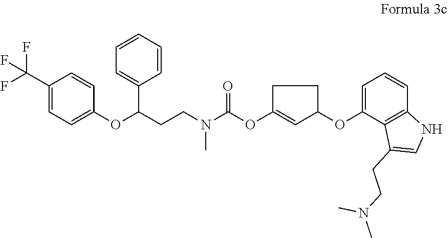 [SMILES: CN(C)CCc1c[nH]c2cccc(OC3C=C(OC(=O)N(C)CCC(Oc4ccc(C(F)(F)F)cc4)c4ccccc4)CC3)c12]

26. The compound of claim 22, wherein the compound is a compound of formula 3d.

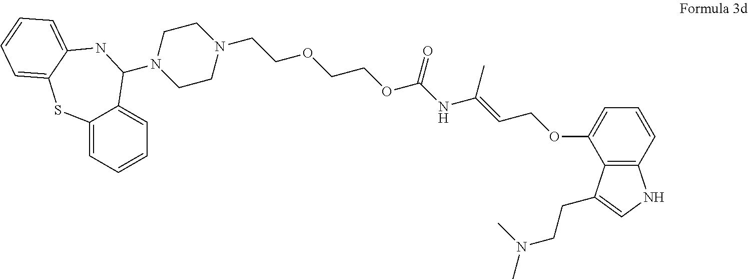 [SMILES: C/C(=C\COc1cccc2[nH]cc(CCN(C)C)c12)NC(=O)OCCOCCN1CCN(C2Nc3ccccc3Sc3ccccc32)CC1]

27. The compound of claim 22, wherein the compound is a compound of formula 3e.

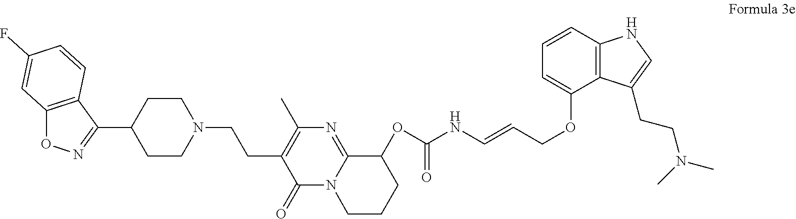 [SMILES: Cc1nc2n(c(=O)c1CCN1CCC(c3noc4cc(F)ccc34)CC1)CCCC2OC(=O)N/C=C/COc1cccc2[nH]cc(CCN(C)C)c12]

28. The compound of claim 22, wherein the compound is a compound of formula 3f.

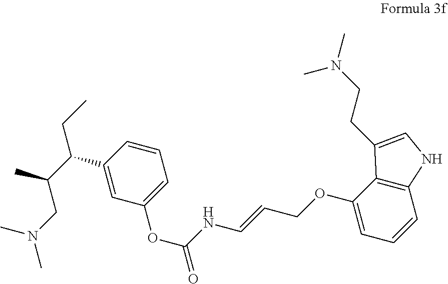 [SMILES: CC[C@H](c1cccc(OC(=O)N/C=C/COc2cccc3[nH]cc(CCN(C)C)c23)c1)[C@H](C)CN(C)C]

29. The compound of claim 22, wherein the compound is a compound of formula 3g.

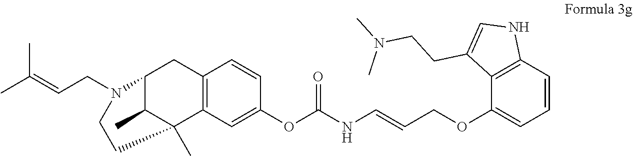 [SMILES: CC(C)=CCN1CC[C@@]2(C)c3cc(OC(=O)N/C=C/COc4cccc5[nH]cc(CCN(C)C)c45)ccc3C[C@@H]1[C@@H]2C]

30. The compound of claim 22, wherein the compound is a compound of formula 3h.

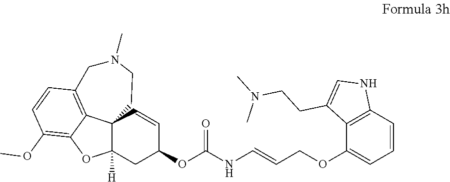 [SMILES: COc1ccc2c3c1O[C@@H]1C[C@H](OC(=O)N/C=C/COc4cccc5[nH]cc(CCN(C)C)c45)C=C[C@]31CCN(C)C2]

31. The compound of claim 22, wherein the compound is a compound of formula 3i.

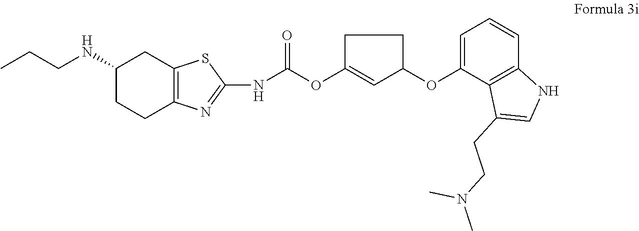 [SMILES: CCCN[C@H]1CCc2nc(NC(=O)OC3=CC(Oc4cccc5[nH]cc(CCN(C)C)c45)CC3)sc2C1]

32. The compound of claim 1, wherein the compound is a compound of formula 4; PS is psilocybin; and DR is selected from the group consisting of: CBD, THC-V, fluoxetine, quetiapine, paliperidone, tapentadol, pentazocine, galantamine, and pramipexole.

33. The compound of claim 32, wherein the compound is a compound of formula 4a.

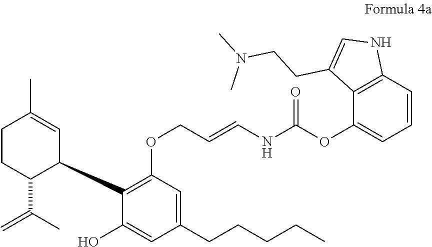 [SMILES: C=C(C)[C@@H]1CCC(C)=C[C@H]1c1c(O)cc(CCCCC)cc1OC/C=C/NC(=O)Oc1cccc2[nH]cc(CCN(C)C)c12]

34. The compound of claim 32, wherein the compound is a compound of formula 4b.

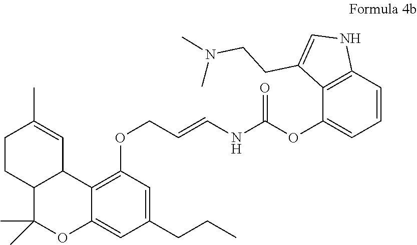 [SMILES: CCCc1cc(OC/C=C/NC(=O)Oc2cccc3[nH]cc(CCN(C)C)c23)c2c(c1)OC(C)(C)C1CCC(C)=CC21]

35. The compound of claim 32, wherein the compound is a compound of formula 4c.

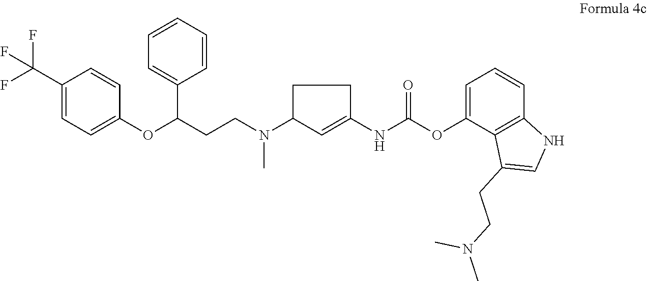 [SMILES: CN(C)CCc1c[nH]c2cccc(OC(=O)NC3=CC(N(C)CCC(Oc4ccc(C(F)(F)F)cc4)c4ccccc4)CC3)c12]

36. The compound of claim 32, wherein the compound is a compound of formula 4d.

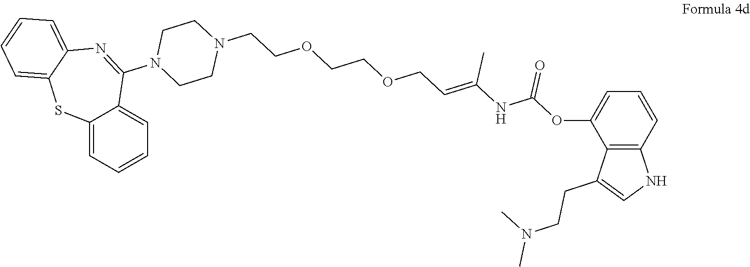 [SMILES: C/C(=C\COCCOCCN1CCN(C2=Nc3ccccc3Sc3ccccc32)CC1)NC(=O)Oc1cccc2[nH]cc(CCN(C)C)c12]

37. The compound of claim 32, wherein the compound is a compound of formula 4e.

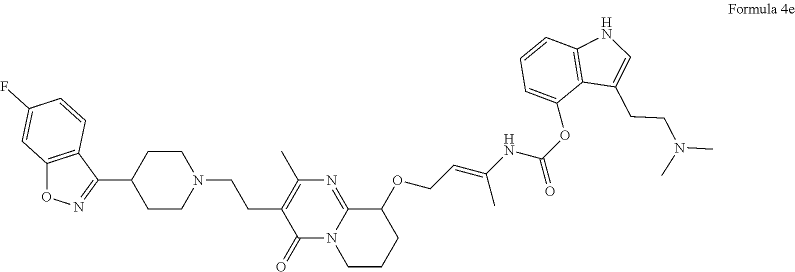 [SMILES: C/C(=C\COC1CCCn2c1nc(C)c(CCN1CCC(c3noc4cc(F)ccc34)CC1)c2=O)NC(=O)Oc1cccc2[nH]cc(CCN(C)C)c12]

38. The compound of claim 32, wherein the compound is a compound of formula 4f.

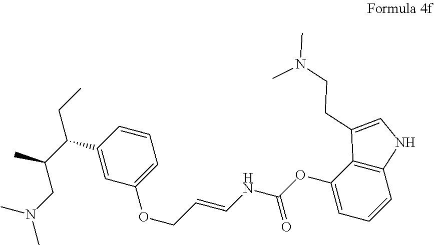 [SMILES: CC[C@H](c1cccc(OC/C=C/NC(=O)Oc2cccc3[nH]cc(CCN(C)C)c23)c1)[C@H](C)CN(C)C]

39. The compound of claim 32, wherein the compound is a compound of formula 4g.

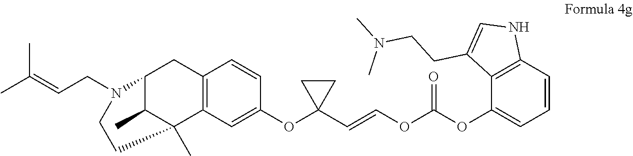 [SMILES: CC(C)=CCN1CC[C@@]2(C)c3cc(OC4(/C=C/OC(=O)Oc5cccc6[nH]cc(CCN(C)C)c56)CC4)ccc3C[C@@H]1[C@@H]2C]

40. The compound of claim 32, wherein the compound is a compound of formula 4h.

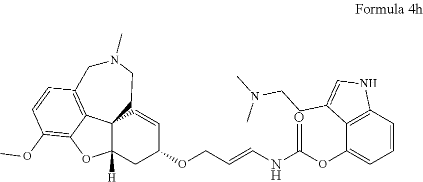 [SMILES: COc1ccc2c3c1O[C@H]1C[C@@H](OC/C=C/NC(=O)Oc4cccc5[nH]cc(CCN(C)C)c45)C=C[C@@]31CCN(C)C2]

41. The compound of claim 32, wherein the compound is a compound of formula 4i.

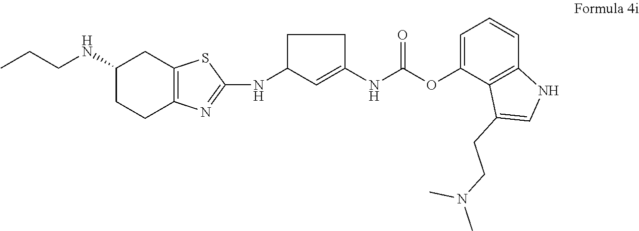 [SMILES: CCCN[C@H]1CCc2nc(NC3C=C(NC(=O)Oc4cccc5[nH]cc(CCN(C)C)c45)CC3)sc2C1]

42. The compound of claim 1, wherein the compound is a compound of formula 5; PS is psilocybin; and each DR is, independently, selected from the group consisting of: CBD, THC-V, fluoxetine, quetiapine, paliperidone, tapentadol, pentazocine, galantamine, carbidopa, gabapentin, nalbuphine, levodopa, and baclofen.

43. The compound of claim 42, wherein the compound is a compound of formula 5a.

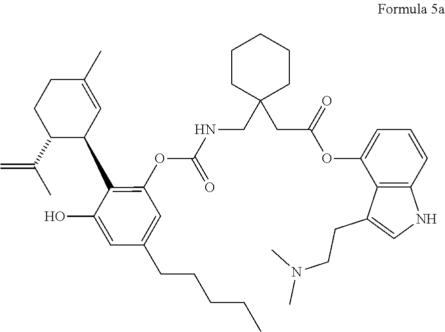 [SMILES: C=C(C)[C@@H]1CCC(C)=C[C@H]1c1c(O)cc(CCCCC)cc1OC(=O)NCC1(CC(=O)Oc2cccc3[nH]cc(CCN(C)C)c23)CCCCC1]

44. The compound of claim 42, wherein the compound is a compound of formula 5b.

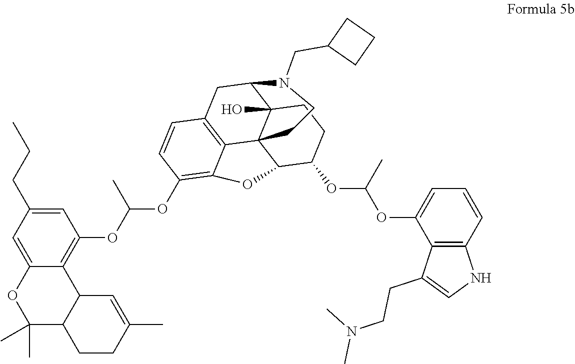 [SMILES: CCCc1cc(OC(C)Oc2ccc3c4c2O[C@H]2[C@@H](OC(C)Oc5cccc6[nH]cc(CCN(C)C)c56)CC[C@@]5(O)[C@@H](C3)N(CC3CCC3)CC[C@]425)c2c(c1)OC(C)(C)C1CCC(C)=CC21]

45. The compound of claim 42, wherein the compound is a compound of formula 5c.

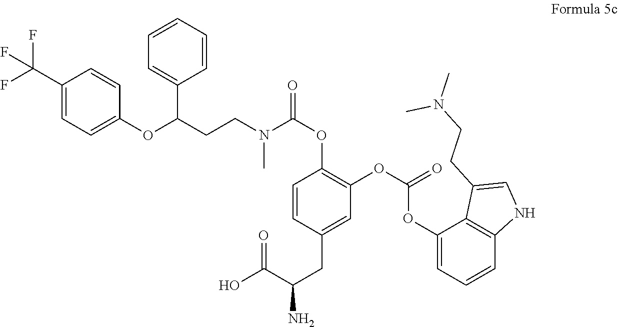 [SMILES: CN(C)CCc1c[nH]c2cccc(OC(=O)Oc3cc(C[C@@H](N)C(=O)O)ccc3OC(=O)N(C)CCC(Oc3ccc(C(F)(F)F)cc3)c3ccccc3)c12]

46. The compound of claim 42, wherein the compound is a compound of formula 5d.

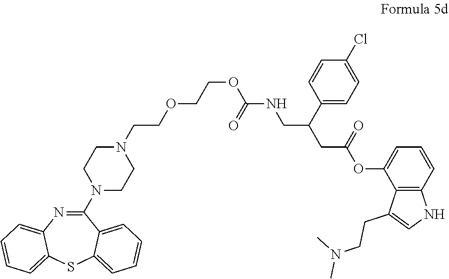 [SMILES: CN(C)CCc1c[nH]c2cccc(OC(=O)CC(CNC(=O)OCCOCCN3CCN(C4=Nc5ccccc5Sc5ccccc54)CC3)c3ccc(Cl)cc3)c12]

47. The compound of claim 42, wherein the compound is a compound of formula 5e.

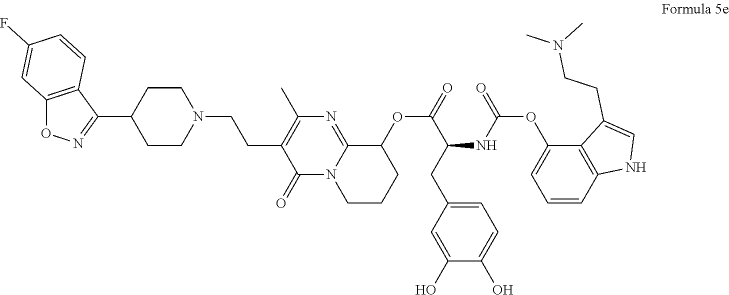 [SMILES: Cc1nc2n(c(=O)c1CCN1CCC(c3noc4cc(F)ccc34)CC1)CCCC2OC(=O)[C@H](Cc1ccc(O)c(O)c1)NC(=O)Oc1cccc2[nH]cc(CCN(C)C)c12]

48. The compound of claim 42, wherein the compound is a compound of formula 5f.

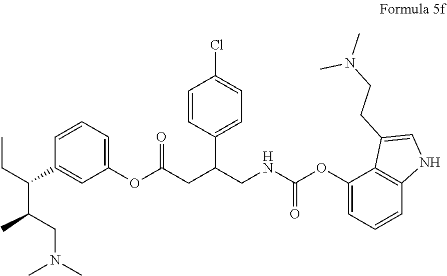 [SMILES: CC[C@H](c1cccc(OC(=O)CC(CNC(=O)Oc2cccc3[nH]cc(CCN(C)C)c23)c2ccc(Cl)cc2)c1)[C@H](C)CN(C)C]

49. The compound of claim 42, wherein the compound is a compound of formula 5g.

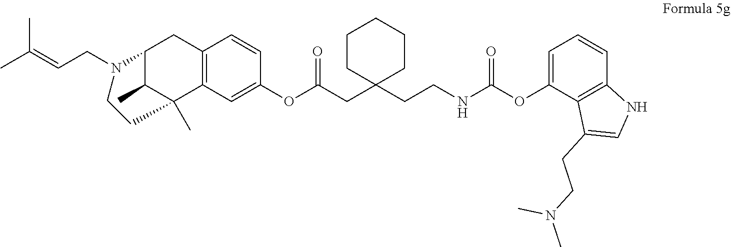 [SMILES: CC(C)=CCN1CC[C@@]2(C)c3cc(OC(=O)CC4(CCNC(=O)Oc5cccc6[nH]cc(CCN(C)C)c56)CCCCC4)ccc3C[C@@H]1[C@@H]2C]

50. The compound of claim 42, wherein the compound is a compound of formula 5h.

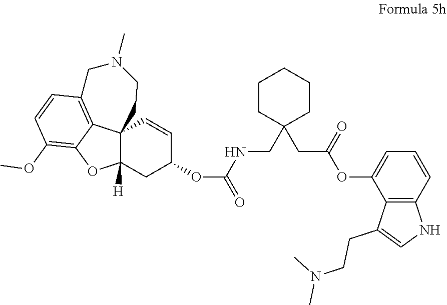 [SMILES: COc1ccc2c3c1O[C@H]1C[C@@H](OC(=O)NCC4(CC(=O)Oc5cccc6[nH]cc(CCN(C)C)c56)CCCCC4)C=C[C@@]31CCN(C)C2]

51. The compound of claim 42, wherein the compound is a compound of formula 5i.

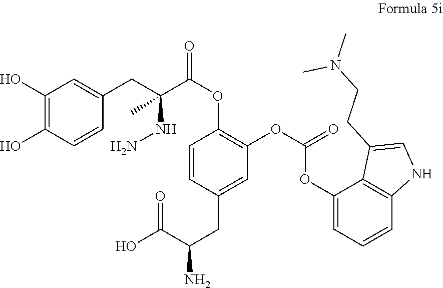 [SMILES: CN(C)CCc1c[nH]c2cccc(OC(=O)Oc3cc(C[C@@H](N)C(=O)O)ccc3OC(=O)[C@](C)(Cc3ccc(O)c(O)c3)NN)c12]

52. The compound of claim 1, wherein the compound is a compound of formula 6; PS is psilocybin; and DR is selected from the group consisting of: CBD, THC-V, fluoxetine, quetiapine, paliperidone, tapentadol, pentazocine, galantamine, and pramipexole.

53. The compound of claim 52, wherein the compound is a compound of formula 6a.

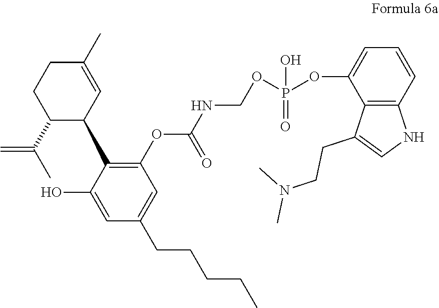 [SMILES: C=C(C)[C@@H]1CCC(C)=C[C@H]1c1c(O)cc(CCCCC)cc1OC(=O)NCOP(=O)(O)Oc1cccc2[nH]cc(CCN(C)C)c12]

54. The compound of claim 52, wherein the compound is a compound of formula 6b.

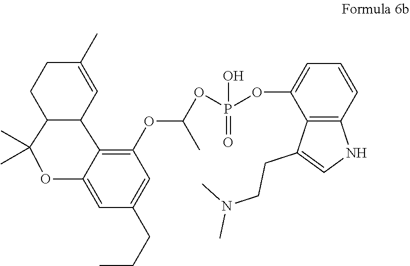 [SMILES: CCCc1cc(OC(C)OP(=O)(O)Oc2cccc3[nH]cc(CCN(C)C)c23)c2c(c1)OC(C)(C)C1CCC(C)=CC21]

55. The compound of claim 52, wherein the compound is a compound of formula 6c.

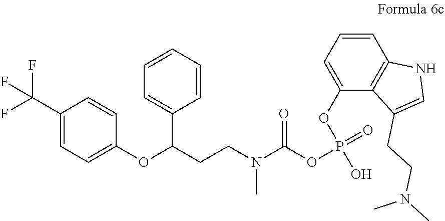 [SMILES: CN(C)CCc1c[nH]c2cccc(OP(=O)(O)OC(=O)N(C)CCC(Oc3ccc(C(F)(F)F)cc3)c3ccccc3)c12]

56. The compound of claim 52, wherein the compound is a compound of formula 6d.

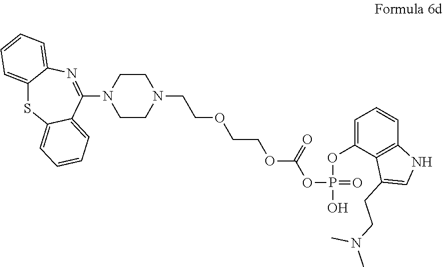 [SMILES: CN(C)CCc1c[nH]c2cccc(OP(=O)(O)OC(=O)OCCOCCN3CCN(C4=Nc5ccccc5Sc5ccccc54)CC3)c12]

57. The compound of claim 52, wherein the compound is a compound of formula 6e.

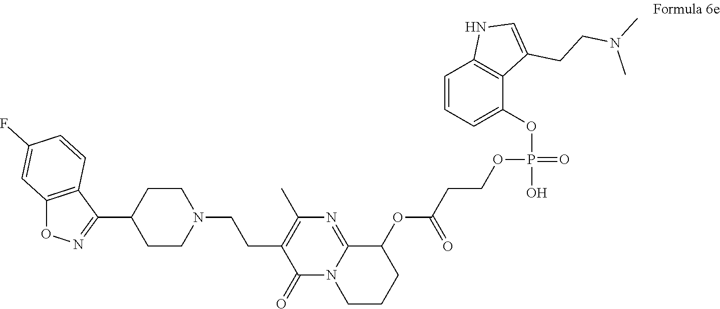 [SMILES: Cc1nc2n(c(=O)c1CCN1CCC(c3noc4cc(F)ccc34)CC1)CCCC2OC(=O)CCOP(=O)(O)Oc1cccc2[nH]cc(CCN(C)C)c12]

58. The compound of claim 52, wherein the compound is a compound of formula 6f.

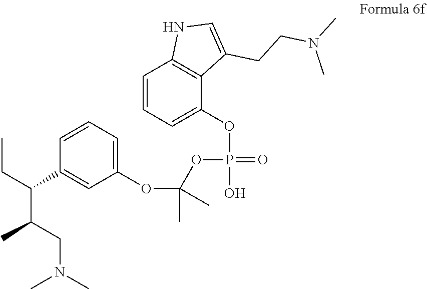 [SMILES: CC[C@H](c1cccc(OC(C)(C)OP(=O)(O)Oc2cccc3[nH]cc(CCN(C)C)c23)c1)[C@H](C)CN(C)C]

59. The compound of claim 52, wherein the compound is a compound of formula 6g.

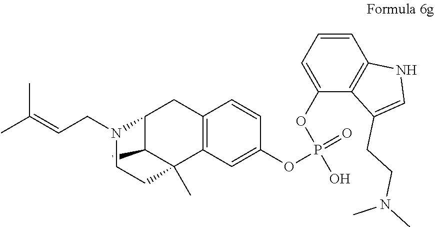 [SMILES: CC(C)=CCN1CC[C@@]2(C)c3cc(OP(=O)(O)Oc4cccc5[nH]cc(CCN(C)C)c45)ccc3C[C@@H]1[C@@H]2C]

60. The compound of claim 52, wherein the compound is a compound of formula 6h.

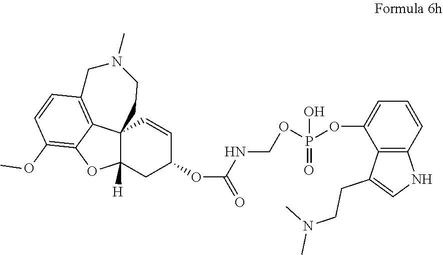 [SMILES: COc1ccc2c3c1O[C@H]1C[C@@H](OC(=O)NCOP(=O)(O)Oc4cccc5[nH]cc(CCN(C)C)c45)C=C[C@@]31CCN(C)C2]

61. The compound of claim 52, wherein the compound is a compound of formula 6i.

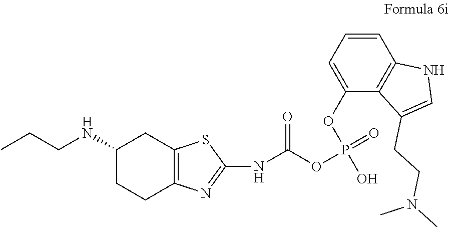 [SMILES: CCCN[C@H]1CCc2nc(NC(=O)OP(=O)(O)Oc3cccc4[nH]cc(CCN(C)C)c34)sc2C1]

62. The compound of claim 1, wherein each DR is, independently, selected from the group consisting of: pregabalin, gabapentin, topiramate, morphine, levodopa, selective serotonin reuptake inhibitor (SSRI), citalopram, amisulpride, lurasidone, paliperidone, paliperidone palmitate, tapentadol, pentazocine, carbidopa, nalbuphine, risperidone, ziprasidone, perospirone, doxorubicin, melperone, aripiprazole, brexpiprazole, cariprazine, olanzapine, quetiapine, fluoxetine, calcitonin, pseudoephedrine, piracetam, levetiracetam, sitagliptin, silodosin, hydrochlorothiazide, ezetimibe, propranolol, atenolol, nadolol, pindolol, sotalol, timolol, penbutolol, oxprenolol, carvedilol, carteolol, bucindolol, acebutanol, betaxolol, esmolol, nebivolol, bisoprolol, celiprolol, metoprolol, azelnidipine, barnidipine, manidipine, lercandipine, efonidipine, benidipine, brimonidine, bortezomib, ledipasvir, daclatasvir, ombitasvir, elbasvir, lamivudine, dopamine, 5-hydroxytryptamine, levodopa, baclofen, pramipexole, ropinirole, rotigotine, apomorphine, tacrine, rivastigmine, donepezil, galantamine, vigabatrin, lamotrigine, tiagabine, amitriptyline, nortriptyline, histamine, and a cannabinoid.

63. The compound of claim 1, wherein each DR is, independently, a cannabinoid selected from the group consisting of: delta-9-tetrahydrocannabinol (THC), delta-8-tetrahydrocannabinol (THC), cannabidiol (CBD), cannabinol (CBN), cannabinolic acid (CBNA), cannabigerol (CBG), cannabigerol (CBG), cannabigerovarin (CBGV), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), cannabigerolic acid monoethyl ether (CBGAM), cannabidiolic acid (CBDA), cannabigerovarinic (CBGVA), cannabichromenic acid (CBCA), cannabichromenic acid (CBCA), cannabidiol monomethylether (CBDM), cannabidiol-C4 (CBD-C4), cannabidivarinic (CBDVA), cannabidiorcol (CBD-C1), delta-9-tetrahydrocannabinolic acid A (THCA-A), delta-9-tetrahydrocannabinolic acid B (THCA-B), delta-9-tetrahydrocannabinolic acid-C4 (THCA-C4), delta-8-tetrahydrocannabinolic acid (delta-8-THCA), delta-8-tetrahydrocannabinol (delta-8-THC), delta-9-tetrahydrocannabinol-C4 (THC-C4), delta-9-tetrahydrocannabiorcolic acid (THCA-C1), delta-9-tetrahydrocannabiorcol-C1 (THC-C1), tetrahydrocannabivarinic acid (THCVA), cannabicycolic acid (CBLA), cannbicyclol (CBL), cannabicyclovarin (CBLV), cannabielsoic acid A (CBEA-A), cannabielsoic acid B (CBEA-B), cannabielsoin (CBE), cannabivarin, cannabinol-C4 (CBN-C4), cannabinol methylether (CBNM), cannabiorcol (CBN-C1), cannabinol-C2 (CBN-C2), cannabinodiol (CBND), cannabinodivarin (CBVD), cannabitriol (CBT), cannabitriolvarin (CBTV), dehydrocannabifuran (DCBF), cannabifuran, cannabicitran (CBT), cannabiripsol (CBR), ‘11-hydroxytetrahydrocannabinol’ (11-OH-THC), ‘11-nor-9-carboxy-tetrahydrocannabinol’ (THC-COOH), and their derivatives, synthetic analogues, related chemical structures, and pharmaceutically acceptable salts.

64. The compound of claim 1, wherein each DR is, independently, CBD or THC-V.

65. The compound of claim 64, wherein PS is selected from the group consisting of: psilocin, psilocybin, baeocystin, norbaeocystin, bufotenine, aeruginascin, a 1H-indazole analogue, a 3-propyl-1H-indole analogue, and an azaindole analogue.

66. The compound of claim 65, wherein one or more halogen groups are added to the carbon backbone of the SPACER.

67. The compound of claim 65, wherein one ore more alkyl groups are added to the carbon backbone of the SPACER.
